(19) United States
(12) Patent Application Publication
(10) Pub. No.: US 2012/0149708 A1
(43) Pub. Date: Jun. 14, 2012

(54) MODULATORS OF VIRAL TRANSCRIPTION, AND METHODS AND COMPOSITIONS THEREWITH

(75) Inventor: Fatah KASHANCHI, Manassas, VA (US)

(73) Assignee: George Mason University

(21) Appl. No.: 13/329,721

(22) Filed: Dec. 19, 2011

Related U.S. Application Data
(63) Continuation-in-part of application No. 13/162,832, filed on Jun. 17, 2011.
(60) Provisional application No. 61/355,711, filed on Jun. 17, 2010.

(51) Int. Cl. A61K 31/53; C12N 9/99; A61P 31/18

(52) U.S. Cl. 514/245; 435/184

(57) ABSTRACT

The present invention is directed to a process for inhibiting the replication of human immunodeficiency virus-1 (HIV-1), by contacting a cell with at least one compound according to Formula I.

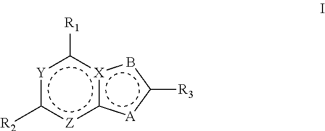

The substituent groups R₁, R₂, R₃, X, Y, Z, A and B are as defined above. Also contemplated is a method for treating or preventing a HIV-1 infection in a subject, by administering a therapeutically effective amount of at least one compound according to Formula I, as well as a method for modulating the activity of a cyclin dependent kinase (cdk) in a cell infected with HIV-1 using a Formula I compound.

### CROSS-REFERENCE TO RELATED PATENT APPLICATIONS

This application is a Continuation-In-Part of U.S. application Ser. No. 13/162,832, filed Jun. 17, 2011, incorporated herein by reference in its entirety, which claims priority from Provisional U.S. Application 61/355,711, filed Jun. 17, 2010, incorporated herein by reference in its entirety.

### STATEMENT REGARDING FEDERALLY SPONSORED RESEARCH

This invention was made with government support under Grant Number 5R21AI065236-02, 1R21AI065236-01A1 awarded by the National Institute of Health (NIH). The United States government has certain rights in the invention.

### BACKGROUND OF THE INVENTION

The present invention relates generally to the field of small molecule therapeutics for treating human immunodeficiency viral (HIV) infections. Human immunodeficiency virus-1 (HIV-1) is etiological agent of acquired immunodeficiency syndrome (AIDS). While clinically used antiretroviral therapy has shown promise in HIV-1 treatment, there are associated drawbacks that are cured by the present invention.

For instance, one drawback of current retroviral therapies is that latently infected cells continue to produce viral RNA and even small amounts of infectious virus. There is a distinct probability, therefore, of viral escape and/or mutational changes in infected cells even after exposure to drug. Current HIV-1 therapies, therefore, are mostly ineffective at eliminating the virus and also are the main cause of drug-resistant viral variants.

Eukaryotic cells possess molecular machinery capable of targeting and destroying small ribonucleic acid (RNA) strands. For instance, RNA interference (RNAi) is a regulatory mechanism conserved in higher eukaryotes that moderates the activity of genes. Two types of small ribonucleic acid (RNA) molecules—microRNA (miRNA) and small interfering RNA (siRNA) are central to RNA interference.

RNAi involves small RNA molecules that guide a protein effecter complex to a complementary or mostly complementary sequence of nucleic acid. The end result is the down regulation of protein expression through either transcriptional silencing, cleavage of target mRNA or inhibition of translation (Agrawal et al, 2003; Bartel, 2004).

The RNAi pathway is initiated by the enzyme Dicer, which cleaves long double-stranded RNA (dsRNA) molecules into short fragments of ˜20 nucleotides that are called siRNAs. For example, exogenously introduced dsRNA is recognized by Dicer, and cleaved into characteristic 21 nucleotide segments with 2 nucleotide 3′overhangs (siRNAs and microRNAs) that direct the RNAi machinery for sequence-specific inhibition of mRNA expression.

Alternatively, microRNAs are produced from genomic DNA that is transcribed by RNA polymerase II. Endogenously expressed RNA can be involved in RNAi through a slightly different pathway involving Droshamediated cleavage of RNA stem-loops in the nucleus, followed by export to the cytoplasm by Exportin-5, and finally cleavage by Dicer to generate a small RNA duplex approximately 22 nucleotides in length with a two nucleotide 3′ overhang on each strand (Hannon, 2002). One strand of the microRNA duplex is incorporated into Argonaute-containing effector complexes, which silence gene expression through two distinct mechanisms. In the first, the small RNA associates with the RNA-induced silencing complex (RISC) and guides the complex to a complementary sequence of mRNA where a member of the Argonaute family of proteins cleave the target mRNA, leading to silencing of a gene. Alternatively, the microRNA may guide the RISC complex to a somewhat complementary region in the 3′UTR of the mRNA. In addition to attaching to the RISC complex, the RNA can associate with the RNA-induced initiation of transcriptional silencing (RITS) complex. Similar to the RISC mechanism, the microRNA guides this complex to a complementary region of chromosomal DNA and recruits factors that modify the chromatin structure and induce transcriptional silencing (Volpe et al, 2002; Matzke and Birchler, 2005).

Several viruses encoding microRNAs have already been identified, including human cytomegalovirus, human herpesevirus 8, Epstein Barr virus, and herpes simplex virus (Grey et al, 2005; Pfeffer et al, 2005; Umbach et al, 2008). The functions of a number of viral microRNA have been dissected and they appear capable of regulating both viral and cellular genes (Dykxhoorn, 2007). In terms of HIV-1, several previous studies have reported the production of microRNAs from the TAR, miR-H1, nef and env RNAs (Omoto et al, 2004; Provost et al, 2006; Klase et al, 2007; Kaul et al, 2009). Because all or few of the HIV-1 generated microRNA could potentially inhibit viral replication, block translation of viral proteins, or cause remodeling of the viral genome, RNAi-based strategies have considerable therapeutic potential against HIV-1 infection.

The majority of current therapies target viral proteins. There is a need, therefore, for development of host gene-based therapies to treat HIV-1 infections. The present invention addresses this need.

### SUMMARY OF THE INVENTION

The present invention is in the field of HIV treatment and prevention of viral replication using small molecules therapeutics. In one embodiment the present invention focuses on a process for inhibiting the replication of human immunodeficiency virus-1 (HIV-1), by contacting a cell with at least one compound according to Formula I or a pharmaceutically acceptable salt, stereoisomer, tautomer, or prodrug thereof.

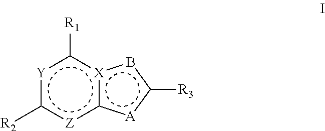

For Formula I compounds, R₁ and R₂ are each independently selected from the group consisting of —H, straight or branched chain (C₁-C₆)alkyl, straight or branched chain (C₁-C₆)hydroxyalkyl, (C₂-C₆)alkene, (C₃-C₈)cycloalkyl, (C₃-C₁₄)aryl, halogen, —NR^{a}R^{b}, —NR^{a}(C₁-C₆)hydroxyalkyl, —NR^{a}(C₁-C₆)alkylene-(C₃-C₁₄)aryl, —NR^{a}(C₁-C₆)alkylene-(C₃-C₁₄)heteroaryl, —NR^{a}(C₁-C₆)alkylene-(C₃-C₁₄)heterocycloalkyl, —NR^{a}(C₁-C₆)alkylene-(C₃-C₁₄)cycloalkyl, —OR', and —SR^{c}.

R₃ is selected from the group consisting of hydrogen, straight or branched chain (C₁-C₆)alkyl, —OH and halogen. Substituent groups X, Y, Z, A and B in Formula I are each independently selected from the group consisting of a bond, —C(R′″)₂—, —CR′″—, —NR′″—, —N—, —O—, —C(O)—, and —S—, with no more than any three of X, Y, Z, A or B simultaneously representing a bond. To avoid the formation of peroxides (—O—O—) and disulfides (—S—S—), for a Formula I compound X and B cannot simultaneously be —O—, or —S—.

To account for the presence of aromatic and non-aromatic ring systems, Formula I recites [symbol] to represent the option of having one or more double bonds.

R^{a}, R^{b}, R^{c} and R′″ in Formula I are each independently selected from the group consisting of H, OH, straight or branched chain (C₁-C₈)alkyl, (C₃-C₆)aryl, —NH₂, —C(O)(C₁-C₆)alkyl, —C(O)(C₃-C₁₄)aryl, (C₃-C₆)cycloalkyl, (C₃-C₁₄)heterocycloalkyl, (C₃-C₁₄)heteroaryl, (C₃-C₁₄)aryl-(C₁-C₆)alkylene-, (C₃-C₆)cycloalkyl-(C₁-C₆)alkylene-, (C₃-C₁₄)heteroaryl-(C₁-C₆)alkylene-, (C₃-C₁₄)heterocycloalkyl-(C₁-C₆)alkylene-.

Further, for compounds that conform to Formula I, any alkyl, alkylene, alkene, aryl, heteroaryl, cycloalkyl, or heterocycloalkyl is optionally substituted with one or more members selected from the group consisting of halogen, oxo, —COOH, —CN, —NO₂, —OH, straight or branched chain (C₁-C₆)alkyl, (C₂-C₆)alkene, (C₃-C₁₄)aryl, (C₃-C₁₄)heteroaryl, (C₃-C₁₄)heterocycloalkyl, (C₃-C₁₄)cycloalkyl, (C₁-C₆)alkoxy, and (C₃-C₁₄)aryloxy.

According to another embodiment, the inventive process calls for a Formula I compound with each of X, Y, Z and B independently being a nitrogen (—N—), substituent A is a C(R′″), and R₃ is hydrogen. Here too, [symbol] represents the option of having one or more double bonds.

For certain Formula I compounds, R′″ is a straight or branched chain (C₁-C₆)alkyl, for example, methyl, ethyl, propyl, or isopropyl. Substituent R₁ in Formula I in one embodiment is NR^{a}(C₁-C₆)alkylene-(C₃-C₁₄)aryl, for example a —NH—(CH₂)-phenyl. In one embodiment, the phenyl is further substituted by a (C₃-C₁₄)heteroaryl, for example, a pyridine.

According to one embodiment, the inventive process recites a Formula I compound that is selected from the following table:

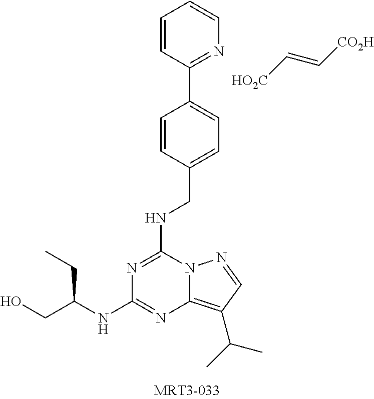

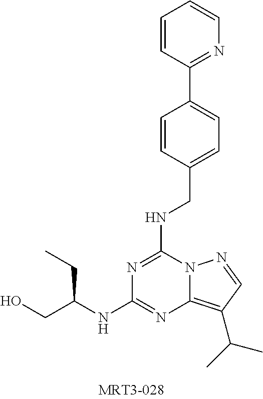

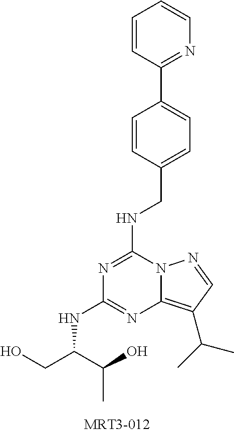

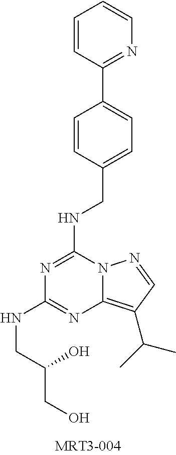

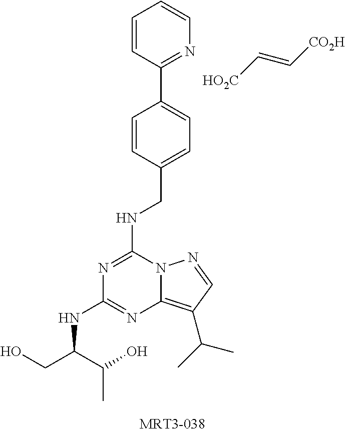

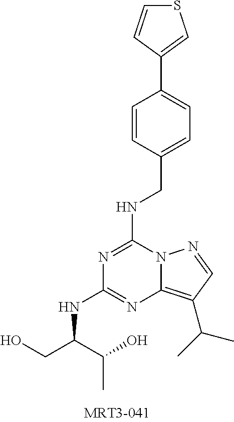

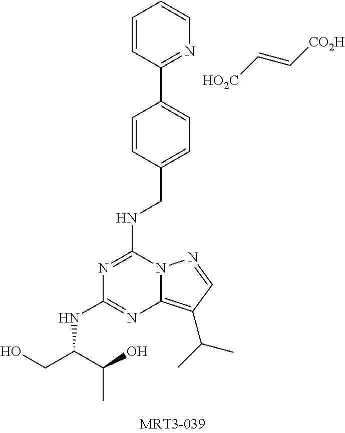

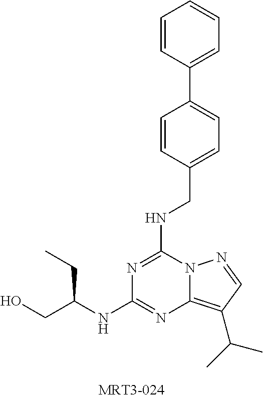

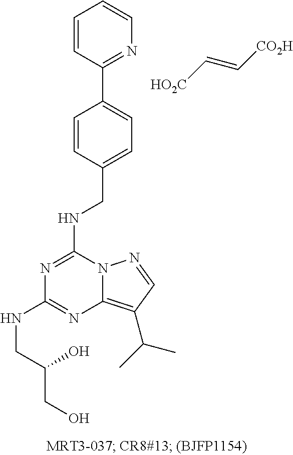

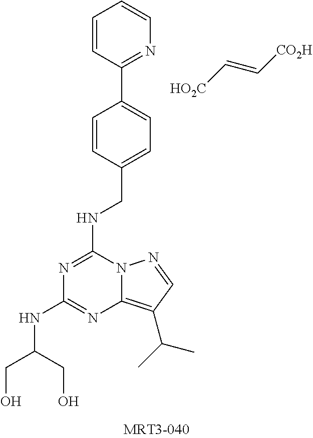

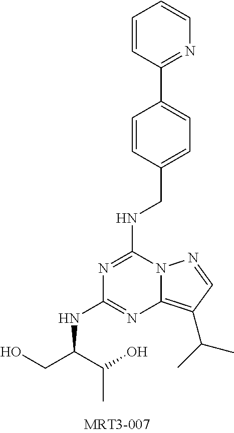

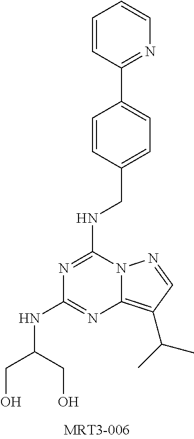

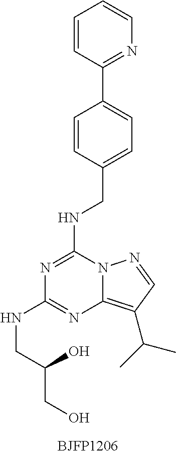

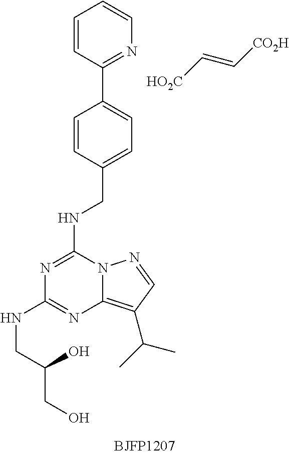

In yet another embodiment, the present invention provides a method for the treatment or prevention of a HIV-1 infection in a subject, comprising administering to the subject therapeutically effective amount of at least one compound according to Formula I.

According to yet another embodiment is provided a method for modulating the activity of a cyclin dependent kinase (cdk) in a cell infected with HIV-1, comprising contacting the cell with a therapeutically effective amount of at least one compound according to Formula I.

### BRIEF DESCRIPTION OF THE DRAWINGS

FIGS. 1 and 2 show screening results of CR8 derivatives on Tat-dependent transcription HIV-1 LTR. FIG. 1 (panel A) is a bar graph demonstrating raw luciferase units with 18 CR8 derivatives. TZM-bl cells were transfected with 1 ug of Tat and treated the next day with DMSO, or the indicated CR8 derivative compounds at 50 nm. 48 hrs-post drug treatment, luciferase activity of the firefly luciferase was measured with the BrightGlo Luciferase Assay and luminescence was read from a 96 well plate on an EG&G Berthold luminometer. Assays were performed in triplicate, average and standard deviations are shown. FIG. 2 (panel B) shows a bar graph demonstrating percent viability based on MTT assays with two different doses (50 nm and 10 μM) of DMSO, CR8, MRT-033, and BJFP1154 (CR8#13) tested on infected ACH2, J1.1, OM10.1, U1 and uninfected CEM, Jurkat T cells, and U937 cells.

FIGS. 3-5 show western blots demonstrating the effect of drugs on Tat-mediated transactivation in HCT116 WT and HCT116 Dicer−/−cells. pHIV-1 LTR-CAT (1 μg) construct was transfected in 2×106 cells in the absence or presence of Tat (1 μg). Six hours later, the transfected cells were treated with DMSO, Flavopirodol (100 nm), CR8#13 (100 nm), F07#13 (100 nm), and 9AA (1000 nm). Treated cells were incubated in complete DMEM for 48 hrs at 37° C. Cells were harvested and cell extracts were used for CAT analysis. One tenth the amount of HCT116 Dicer−/−extract compared to HCT116 WT was used for CAT analysis. FIG. 3 (panel A) is a western blot that shows results from HCT116 WT cells and panel and FIG. 3 (panel B) is a western blot that shows results from HCT116 Dicer—/—cells. The corresponding bar graphs in A and B show values that represent the percentage of conversion of the [14C] chloramphenicol substrate in the CAT assay.

FIG. 4 (panel C) is a western blot for Dicer and PIWIL4 in the HCT116 WT and Dicer−/−cells. One hundred micrograms of total extracts were run on a 4-20% (w/v) SDS/PAGE and western blotted for presence of Dicer, PIWIL4 and actin.

FIG. 5 (panel D) is a western blot from a CAT assay of Dicer WT HCT116 cells that had been treated with siRNA against Dicer, transfection with Tat followed by drug treatment.

FIGS. 6-8 (panels A-C) are western blots and a bar graph. FIG. 6 (panel A) is a western blot (50 μg of total protein) for Dicer, Drosha, Ago2 and PIWIL4 in control T-cells (CEM) and monocytes (U937). Dicer protein expression becomes apparent only after PMA treatment resulting in differentiation of cells into macrophages. PIWIL4 are present in both cell types. FIG. 7 (panel B) is a western blot of monocytes (U937) and monocytes (U937) treated with PMA.

FIG. 8 (panel C) is a graphical representation demonstrating results from reverse transcriptase (RT) reaction assays to determine virus production in drug treated cells. Jurkat T-cell and promonocytic U937 cells were electroporated with 5 μg pNL4.3 followed the next day by drug treatment of Flavopiridol (200 nm), CR8 (100 nm) or CR8#13 (50 nm). Cell supernatants were collected at 48 hours post drug treatment. Viral supernatants (10 μl) were incubated in a 96-well plate with reverse transcriptase (RT) reaction mixture overnight at 37° C., and 10 μl of the reaction mix was spotted on a DEAE Filtermat paper, washed with 5% (w/v) Na2HPO4 followed by water wash, and then dried completely. RT activity was measured in a Betaplate counter.

FIG. 9 shows bands from agarose gels demonstrating a lack of effect on cellular genes controlled by cdk9 after treatment with CR8#13. 293T cells were treated with three different concentrations of CR8#13 (20 nm, 50 nm, and 200 nm). Cells were processed 48 hrs-post treatment for RT-PCR. Effector cdk9 genes such as CIITA, IL-8, CAD, MCL-1, Cyclin D1, and PBX-1 were used in the RT-PCR.

FIGS. 10-14: FIG. 10 (panel A) is a model of the effect of RNA polymerase II phosphorylation on transcription. RNA polymerase II CTD is hypo-phosphorylated at the initiation complex; SerS is only phosphorylated at the promoter clearance stage; and Ser2 is mostly phosphorylated at the elongation phase. HIV-1 genome is unique in that it contains both Ser2 and SerS phosphorylation at the elongation stage (Zhou et al, 2004). Phosphorylation of Ser2 and Ser5 could be seen by multiple cyclin/cdk complexes.

FIG. 11 (panel B) are bands from a gel demonstrating small RNA fragments corresponding to TAR sequence from RNase protection assays. Ten micrograms of total RNA from TNF treated CEM (lane 1) and TNF treated ACH2 cells (lanes 2-6) were hybridized to a radiolabeled TAR 5′ probe and then treated with RNase A. Arrows indicate the probe protected by TAR at 27 nucleotides and the probe protected by a TAR miRNA at approximately 22 nt. Cyc202 concentration at 500 nm, CR8 at 100 nm, CR8#13 at 50 nm, and Flavopiridol at 50 nm were used for these experiments.

FIG. 12 (panel C) are bands from a gel demonstrating results from ACH2 cells that were treated with Flavopiridol (50 nm), CR8 (100 nm) and CR8#13 (100 nm). RNA was extracted 48 hrs-post drug treatment. 500 ng of RNA from the microRNA-enriched fraction was used to generate cDNA using the Quantimir kit (SBI). RT reactions are performed followed by PCR in which a universal reverse primer is provided by the manufacturer. Specific microRNA forward primers are identical in sequence to the microRNA of interest. PCR products corresponding to the amplified microRNAs were resolved in a 3.5% (w/v) agarose gel. The PCR products are at around 67 bp as compared with the Fermentas 1 kb DNA Plus Ladder. Increased amounts of 3′ and 5′ TAR microRNA were observed post drug treatment.

FIG. 13 (panel D) are bands from a gel demonstrating results from total RNA (1 ug) from each samples was separated in a 1% (w/v) agarose gel. The location of both 18S and 28S are shown.

FIG. 14 (panel E) is a bar graph demonstrating results from a RT assay that was performed to detect viral levels in ACH2 cells after TNF and drug treatments. ACH2 cells were treated with Flavopiridol (50 nm), Cyc202 (500 nm), CR8 (100 nm) and CR8#13 (100 nm). Supernatants were collected 48 hrs later and used for RT assay. TNF treatment significantly increased RT levels in ACH2 cells and drug treatment was able to decrease RT levels.

FIGS. 15-20: FIG. 15 (panel A) is a model of TZM-bl cells suppression and activation. Trichostatin-A (TSA), a widely used HDAC inhibitor were used to activate the integrated HIV-1 LTRLuc transcription in TZM-bl and abolish the repressive heterochromatic state. Seven days post treatment of TSA, the TZM-bl were transfected with the TAR microRNA.

FIG. 16 (panel B) are bands from a gel demonstrating chromatin changes using antibodies specific for inhibitory factors verified by ChIP assays performed with the TSA treated TZM-bls. Primers specific for the HIV-1 LTR were used to amplify DNA that was precipitated with each antibody. MicroRNA machinery (Ago2), histone methyltransferases (Suv39H1), chromatin remodeling markers (SETDB1, SETMAR), and transcription repressors (PIASγ) were downregulated after TSA treatment on the integrated HIV-LTR.

FIG. 17 (panel C) are bands from a gel demonstrating results from ChIP assays that were performed on several markers of chromatin repression (HDAC1) and microRNA machinery (Ago2) in TZMbl cells. Primers specific for the HIV-1 LTR were used to amplify DNA that was precipitated with each antibody. Lane 1 shows basal levels of repressive markers on the HIV-1 LTR. Lane 2 shows that seven days of TSA treatment removes the markers of repressive chromatin. Lane 3 shows that the TAR-D mutant does not initiate a recruitment of repressive enzymes. Lane 4 demonstrates that addition of the WT-TAR molecule is sufficient to recruit Ago2 and HDAC1 back to the HIV-1 LTR region.

FIG. 18 (panel D) are bands from a gel demonstrating results from TZM-bl cells that were treated with Flavopiridol (50 nm), CR8 (100 nm) and CR8#13 (100 nm) after 7-day TSA treatment. RNA was extracted 48 hrs-post drug treatment. 500 ng of RNA from the microRNA-enriched fraction was used to generate cDNA using the Quantimir kit (SBI) in order to poly adenylate small RNA species. RT reactions were performed followed by PCR in which a universal reverse primer was provided by the manufacturer. Specific microRNA forward primers are identical in sequence to the microRNA of interest. PCR products corresponding to the amplified microRNAs were separated in a 3.5% (w/v) agarose gel. The PCR products are at around 67 bp as compared with the Fermentas 1 kb DNA Plus Ladder. Increased levels of 3′TAR microRNA were produced post CR8#13 treatment.

FIG. 19 (panel E) are bands from a gel demonstrating results from ChIP assays that were performed on several markers of chromatin repression (HDAC1, Suv39H1) and microRNA machinery (Ago2) in TZMbl cells. Primers specific for the HIV-1 LTR were used to amplify DNA that was precipitated with each antibody. Lane 1 indicates basal levels of repressive markers on the HIV-1 LTR. Lane 2 indicates that seven days of TSA treatment removes the markers of repressive chromatin and Lane 3 shows results that the CR8#13 treatment is sufficient to recruit HDAC1, Ago2 and Suv39H1 back to the HIV-1 LTR region.

FIG. 20 (panel F) is a bar graph demonstrating results from luciferase assays that were performed on the cells used in FIG. 6E. Luciferase activity increased with TSA treatment and then decreased post-CR8#13 treatment.

FIG. 21 depicts a non-limiting non-binding model for cdk inhibitor-mediated viral microRNA production and transcriptional inhibition. During viral transcription, Tat/pTEF-b complexes increase phosphorylation of RNA polymerase II, leading to increased transcriptional elongation. In contrast, cdk inhibitors reduce phosphorylation of RNA polymerase II (at either Ser 2, 5 or both), consequently decreasing elongation. As a result, increased TAR transcripts are produced which aid in the recruitment of RNA interference machinery and heterochromatin remodeling complexes to the HIV-1 promoter, inhibiting transcription. This form of inhibition may ultimately lead to DNA methylation as a permanent epigenetic mark on HIV-1 LTR.

### DETAILED DESCRIPTION OF THE PREFERRED EMBODIMENTS

### Definitions

“Alkyl” refers to straight, branched chain, or cyclic hydrocarbyl groups including from 1 to about 20 carbon atoms. For instance, an alkyl can have from 1 to 10 carbon atoms or 1 to 5 carbon atoms. Exemplary alkyl includes straight chain alkyl groups such as methyl, ethyl, propyl, butyl, pentyl, hexyl, heptyl, octyl, nonyl, decyl, undecyl, dodecyl, and the like, and also includes branched chain isomers of straight chain alkyl groups, for example without limitation, —CH(CH₃)₂, —CH(CH₃)(CH₂CH₃), —CH(CH₂CH₃)₂, —C(CH₃)₃, —C(CH₂CH₃)₃, —CH₂CH(CH₃)₂, —CH₂CH(CH₃)(CH₂CH₃), —CH₂CH(CH₂CH₃)₂, —CH₂C(CH₃)₃, —CH₂C(CH₂CH₃)₃, —CH(CH₃)CH(CH₃)(CH₂CH₃), —CH₂CH₂CH(CH₃)₂, —CH₂CH₂CH(CH₃)(CH₂CH₃), —CH₂CH₂CH(CH₂CH₃)₂, —CH₂CH₂C(CH₃)₃, —CH₂CH₂C(CH₂CH₃)₃, —CH(CH₃)CH₂CH(CH₃)₂, —CH(CH₃)CH(CH₃)CH(CH₃)₂, and the like. Thus, alkyl groups include primary alkyl groups, secondary alkyl groups, and tertiary alkyl groups.

The phrase “substituted alkyl” refers to alkyl substituted at one or more positions, for example, 1, 2, 3, 4, 5, or even 6 positions, which substituents are attached at any available atom to produce a stable compound, with substitution as described herein. “Optionally substituted alkyl” refers to alkyl or substituted alkyl.

Each of the terms “halogen,” “halide,” and “halo” refers to —F, —Cl, —Br, or —I.

The terms “alkylene” and “substituted alkylene” refer to divalent alkyl and divalent substituted alkyl, respectively. Examples of alkylene include without limitation, ethylene (—CH₂—CH₂—). “Optionally substituted alkylene” refers to alkylene or substituted alkylene.

“Alkene” refers to straight, branched chain, or cyclic hydrocarbyl groups including from 2 to about 20 carbon atoms having 1-3, 1-2, or at least one carbon to carbon double bond. “Substituted alkene” refers to alkene substituted at 1 or more, e.g., 1, 2, 3, 4, 5, or even 6 positions, which substituents are attached at any available atom to produce a stable compound, with substitution as described herein. “Optionally substituted alkene” refers to alkene or substituted alkene.

The term “alkenylene” refers to divalent alkene. Examples of alkenylene include h(without limitation, ethenylene (—CH═CH—) and all stereoisomeric and conformational isomeric forms thereof “Substituted alkenylene” refers to divalent substituted alkene. “Optionally substituted alkenylene” refers to alkenylene or substituted alkenylene.

“Alkyne or “alkynyl” refers to a straight or branched chain unsaturated hydrocarbon having the indicated number of carbon atoms and at least one triple bond. An alkynyl group can be unsubstituted or optionally substituted with one or more substituents as described herein below.

The term “alkynylene” refers to divalent alkyne. Examples of alkynylene include without limitation, ethynylene, propynylene. “Substituted alkynylene” refers to divalent substituted alkyne.

The term “alkoxy” refers to an —O-alkyl group having the indicated number of carbon atoms. For example, a (C₁-C₆)alkoxy group includes —O-methyl, —O-ethyl, —O-propyl, —O-isopropyl, —O-butyl, —O-sec-butyl, —O-tert-butyl, —O-pentyl, —O-isopentyl, —O-neopentyl, —O-hexyl, —O-isohexyl, and —O-neohexyl.

The term “aryl,” alone or in combination refers to an aromatic monocyclic or bicyclic ring system such as phenyl or naphthyl. “Aryl” also includes aromatic ring systemts that are optionally fused with a cycloalkyl ring, as herein defined.

A “substituted aryl” is an aryl that is independently substituted with one or more substituents attached at any available atom to produce a stable compound, wherein the substituents are as described herein. “Optionally substituted aryl” refers to aryl or substituted aryl.

“Arylene” denotes divalent aryl, and “substituted arylene” refers to divalent substituted aryl. “Optionally substituted arylene” refers to arylene or substituted arylene.

The term “heteroatom” refers to N, O, and S. Inventive compounds that contain N or S atoms can be optionally oxidized to the corresponding N-oxide, sulfoxide,or sulfone compounds.

“Heteroaryl,” alone or in combination with any other moiety described herein, refers to a monocyclic aromatic ring structure containing 5 or 6 ring atoms, or a bicyclic aromatic group having 8 to 10 atoms, containing one or more, such as 1-4, 1-3, or 1-2, heteroatoms independently selected from the group consisting of O, S, and N. Heteroaryl is also intended to include oxidized S or N, such as sulfinyl, sulfonyl and N-oxide of a tertiary ring nitrogen. A carbon or heteroatom is the point of attachment of the heteroaryl ring structure such that a stable compound is produced. Examples of heteroaryl groups include, but are not limited to, pyridinyl, pyridazinyl, pyrazinyl, quinaoxalyl, indolizinyl, benzo[b]thienyl, quinazolinyl, purinyl, indolyl, quinolinyl, pyrimidinyl, pyrrolyl, pyrazolyl, oxazolyl, thiazolyl, thienyl, isoxazolyl, oxathiadiazolyl, isothiazolyl, tetrazolyl, imidazolyl, triazolyl, furanyl, benzofuryl, and indolyl.

A “substituted heteroaryl” is a heteroaryl that is independently substituted, unless indicated otherwise, with one or more, e.g., 1, 2, 3, 4 or 5, also 1, 2, or 3 substituents, also 1 substituent, attached at any available atom to produce a stable compound, wherein the substituents are as described herein. “Optionally substituted heteroaryl” refers to heteroaryl or substituted heteroaryl.

“Heteroarylene” refers to divalent heteroaryl, and “substituted heteroarylene” refers to divalent substituted heteroaryl. “Optionally substituted heteroarylene” refers to heteroarylene or substituted heteroarylene.

“Heterocycloalkyl” means a saturated or unsaturated non-aromatic monocyclic, bicyclic, tricyclic or polycyclic ring system that has from 5 to 14 atoms in which from 1 to 3 carbon atoms in the ring are replaced by heteroatoms of O, S or N. A heterocycloalkyl is optionally fused with benzo or heteroaryl of 5-6 ring members, and includes oxidized S or N, such as sulfinyl, sulfonyl and N-oxide of a tertiary ring nitrogen. The point of attachment of the heterocycloalkyl ring is at a carbon or heteroatom such that a stable ring is retained. Examples of heterocycloalkyl groups include without limitation morpholino, tetrahydrofuranyl, dihydropyridinyl, piperidinyl, pyrrolidinyl, piperazinyl, dihydrobenzofuryl, and dihydroindolyl.

“Optionally substituted heterocycloalkyl” denotes heterocycloalkyl that is substituted with 1 to 3 substituents, e.g., 1, 2 or 3 substituents, attached at any available atom to produce a stable compound, wherein the substituents are as described herein.

“Heteroalkyl” means a saturated alkyl group having from 1 to about 20 carbon atoms, 1 to 10 carbon atoms, 1 to 6 carbon atoms, or 1 to 3 carbon atoms, in which from 1 to 3 carbon atoms are replaced by heteroatoms of O, S or N. Heteroalkyl is also intended to include oxidized S or N, such as sulfinyl, sulfonyl and N-oxide of a tertiary ring nitrogen. The point of attachment of the heteroalkyl substituent is at an atom such that a stable compound is formed. Examples of heteroalkyl groups include, but are not limited to, N-alkylaminoalkyl (e.g., CH₃NHCH₂—), N,N-dialkylaminoalkyl (e.g., (CH₃)₂NCH₂—), and the like.

“Heteroalkylene” refers to divalent heteroalkyl. The term “optionally substituted heteroalkylene” refers to heteroalkylene that is substituted with 1 to 3 substituents, e.g., 1, 2 or 3 substituents, attached at any available atom to produce a stable compound, wherein the substituents are as described herein.

“Heteroalkene” means a unsaturated alkyl group having from 1 to about 20 carbon atoms, 1 to 10 carbon atoms, 1 to 6 carbon atoms, or 1 to 3 carbon atoms, in which from 1 to 3 carbon atoms are replaced by heteroatoms of O, S or N, and having 1-3, 1-2, or at least one carbon to carbon double bond or carbon to heteroatom double bond.

“Heteroalkenylene” refers to divalent heteroalkene. The term “optionally substituted heteroalkenylene” refers to heteroalkenylene that is substituted with 1 to 3 substituents, e.g., 1, 2 or 3 substituents, attached at any available atom to produce a stable compound, wherein the substituents are as described herein.

The term “cycloalkyl” refer to monocyclic, bicyclic, tricyclic, or polycyclic, 3- to 14-membered ring systems, which are either saturated, unsaturated or aromatic. The heterocycle may be attached via any atom. Cycloalkyl also contemplates fused rings wherein the cycloalkyl is fused to an aryl or hetroaryl ring as defined above. Representative examples of cycloalkyl include, but are not limited to cyclopropyl, cycloisopropyl, cyclobutyl, cyclopentyl, cyclohexyl, cyclopropene, cyclobutene, cyclopentene, cyclohexene, phenyl, naphthyl, anthracyl, benzofuranyl, and benzothiophenyl. A cycloalkyl group can be unsubstituted or optionally substituted with one or more substituents as described herein below.

The term “cycloalkylene” refers to divalent cycloalkylene. The term “optionally substituted cycloalkylene” refers to cycloalkylene that is substituted with 1 to 3 substituents, e.g., 1, 2 or 3 substituents, attached at any available atom to produce a stable compound, wherein the substituents are as described herein.

The term ‘nitrile or cyano” can be used interchangeably and refer to a —CN group which is bound to a carbon atom of a heteroaryl ring, aryl ring and a heterocycloalkyl ring.

The term “oxo” refers to a ═O atom attached to a saturated or unsaturated (C₃-C₈) cyclic or a (C₁-C₈) acyclic moiety. The ═O atom can be attached to a carbon, sulfur, and nitrogen atom that is part of the cyclic or acyclic moiety.

The term “amine or amino” refers to an —NR^{d}R^{e} group wherein R^{d} and R^{e} each independently refer to a hydrogen, (C₁-C₈)alkyl, aryl, heteroaryl, heterocycloalkyl, (C₁-C₈)haloalkyl, and (C₁-C₆)hydroxyalkyl group.

The term “amide” refers to a —NR′R″C(O)-group wherein R′ and R″ each independently refer to a hydrogen, (C₁-C₈)alkyl, or (C₃-C₆)aryl.

The term “carboxamido” refers to a —C(O)NR′R″ group wherein R′ and R″ each independently refer to a hydrogen, (C₁-C₈)alkyl, or (C₃-C₆)aryl.

The term “aryloxy” refers to an —O-aryl group having the indicated number of carbon atoms. Examples of aryloxy groups include, but are not limited to, phenoxy, napthoxy and cyclopropeneoxy.

The term “haloalkoxy,” refers to an —O-(C₁-C₆)alkyl group wherein one or more hydrogen atoms in the C₁-C₈ alkyl group is replaced with a halogen atom, which can be the same or different. Examples of haloalkyl groups include, but are not limited to, difluoromethoxy, trifluoromethoxy, 2,2,2-trifluoroethoxy, 4-chlorobutoxy, 3-bromopropyloxy, pentachloroethoxy, and 1,1,1-trifluoro-2-bromo-2-chloroethoxy.

The term “hydroxyalkyl,” refers to an alkyl group having the indicated number of carbon atoms wherein one or more of the alkyl group's hydrogen atoms is replaced with an —OH group. Examples of hydroxyalkyl groups include, but are not limited to, —CH₂OH, —CH₂CH₂OH, —CH₂CH₂CH₂OH, —CH₂CH₂CH₂CH₂OH, —CH₂CH₂CH₂CH₂CH₂OH, —CH₂CH₂CH₂CH₂CH₂CH₂OH, and branched versions thereof.

The term “haloalkyl,” refers to an (C₁-C₆)alkyl group wherein one or more hydrogen atoms in the C₁-C₆ alkyl group is replaced with a halogen atom, which can be the same or different. Examples of haloalkyl groups include, but are not limited to, difluoromethyl, trifluoromethyl, 2,2,2-trifluoroethyl, 4-chlorobutyl, 3-bromopropylyl, pentachloroethyl, and 1,1,1-trifluoro-2-bromo-2-chloroethyl.

The term “aminoalkyl,” refers to an (C₁-C₆)alkyl group wherein one or more hydrogen atoms in the C₁-C₆ alkyl group is replaced with a —NR^{d}R^{e} group, where R^{d} and R^{e} can be the same or different, for example, R^{d} and R^{e} each independently refer to a hydrogen, (C₁-C₈)alkyl, aryl, heteroaryl, heterocycloalkyl, (C₁-C₈)haloalkyl, (C₃-C₆)cycloalkyl and (C₁-C₆)hydroxyalkyl group. Examples of aminoalkyl groups include, but are not limited to, aminomethyl, aminoethyl, 4-aminobutyl and 3-aminobutylyl.

The term “thioalkyl” or “alkylthio” refers to a (C₁-C₆)alkyl group wherein one or more hydrogen atoms in the C₁-C₆ alkyl group is replaced with a —SR^{j} group, wherein R^{j} is selected from the group consisting of hydrogen, (C₁-C₆)alkyl and (C₃-C₁₄)aryl.

“Amino (C₁-C₆)alkylene” refers to a divalent alkylene wherein one or more hydrogen atoms in the C₁-C₆ alkylene group is replaced with a —NR^{d}R^{e} group. Examples of amino (C₁-C₆)alkylene include, but are not limited to, aminomethylene, aminoethylene, 4-aminobutylene and 3-aminobutylylene.

A “hydroxyl” or “hydroxy” refers to an —OH group.

The term NR^{a}(C₁-C₆)alkylene-(C₃-C₁₄)aryl refers to a amine in which one substituent is a divalent alkylene group to which is group attached a substituted or unsubstituted aryl.

The term NR^{a}(C₁-C₆)alkylene-(C₃-C₁₄)heteroaryl refers to a amine in which one substituent is a divalent alkylene group to which group is attached a substituted or unsubstituted heteroaryl.

The term NR^{a}(C₁-C₆)alkylene-(C₃-C₁₄)aryl refers to a amine in which one substituent is a divalent alkylene group to which group is attached a substituted or unsubstituted heterocycloalkyl.

The term NR^{a}(C₁-C₆)alkylene-(C₃-C₁₄)aryl refers to a amine in which one substituent is a divalent alkylene group to which group is attached a substituted or unsubstituted cycloalkyl.

The term “(C₃-C₁₄)aryl-(C₁-C₆)alkylene” refers to a divalent alkylene wherein one or more hydrogen atoms in the C₁-C₆ alkylene group is replaced by a (C₃-C₁₄)aryl group. Examples of (C₃-C₁₄)aryl-(C₁-C₆)alkylene groups include without limitation 1-phenylbutylene, phenyl-2-butylene, 1-phenyl-2-methylpropylene, phenylmethylene, phenylpropylene, and naphthylethylene.

The term “(C₃-C₁₄)heteroaryl-(C₁-C₆)alkylene” refers to a divalent alkylene wherein one or more hydrogen atoms in the C₁-C₆ alkylene group is replaced a (C₃-C₁₄)heteroaryl group. Examples of (C₃-C₁₄)heteroaryl-(C₁-C₆)alkylene groups include without limitation 1-pyridylbutylene, quinolinyl-2-butylene and 1-pyridyl-2-methylpropylene.

The term “(C₃-C₁₄)heterocycloalkyl-(C₁-C₆)alkylene” refers to a divalent alkylene wherein one or more hydrogen atoms in the C₁-C₆ alkylene group is replaced by a (C₃-C₁₄)heterocycloalkyl group. Examples of (C₃-C₁₄)heterocycloalkyl-(C₁-C₆)alkylene groups include without limitation 1-morpholinopropylene, azetidinyl-2-butylene and 1-tetrahydrofuranyl-2-methylpropylene.

The term “(C₃-C₁₄)heteroaryl-(C₁-C₁₄)hetercycloalkylene” refers to a divalent heterocycloalkylene wherein one or more hydrogen atoms in the C₁-C₆ heterocycloalkylene group is replaced by a (C₃-C₁₄)heteroaryl group. Examples of (C₃-C₁₄)heteroaryl-(C₁-C₆)heterocycloalkylene groups include without limitation pyridylazetidinylene and 4-quinolino-1-piperazinylene.

The term “(C₃-C₁₄)aryl-(C₁-C₁₄)heterocycloalkylene” refers to a divalent heterocycloalkylene wherein one or more hydrogen atoms in the C₁-C₁₄ heterocycloalkylene group is replaced by a (C₃-C₁₄)aryl group. Examples of (C₃-C₁₄)aryl-(C₁-C₁₄)heterocycloalkylene groups include without limitation 1-naphthyl-piperazinylene, phenylazetidinylene, and phenylpiperidinylene.

The term “(C₃-C₁₄)aryl-(C₁-C₆)alkyl-(C₁-C₁₄)heterocycloalkylene” refers to a divalent heterocycloalkylene wherein one or more hydrogen atoms in the C₁-C₁₄ heterocycloalkylene group is replaced by a (C₁-C₆) alkyl group that is further substituted by replacing one or more hydrogen atoms of the (C₁-C₆) alkyl group with a (C₃-C₁₄)aryl group.

The term “(C₃-C₁₄)heteroaryl-(C₁-C₆)alkyl-(C₁-C₁₄)heterocycloalkylene” refers to a divalent heterocycloalkylene wherein one or more hydrogen atoms in the C₁-C₁₄ heterocycloalkylene group is replaced by a (C₁-C₆) alkyl group that is further substituted by replacing one or more hydrogen atoms of the (C₁-C₆) alkyl group with a (C₃-C₁₄)heteroaryl group.

The term “(C₃-C₁₄)heterocycloalkyl-(C₁-C₆)alkyl-(C₁-C₁₄)heterocycloalkylene” refers to a divalent heterocycloalkylene wherein one or more hydrogen atoms in the C₁-C₁₄ heterocycloalkylene group is replaced by a (C₁-C₆) alkyl group that is further substituted by replacing one or more hydrogen atoms of the (C₁-C₆) alkyl group with a (C₃-C₁₄)heterocycloalkyl group.

The term “(C₃-C₁₄)aryl-(C₁-C₁₄)cycloalkylene” refers to a divalent cycloalkylene that is monocyclic, bicyclic or polycyclic and wherein one or more hydrogen atoms in the (C₁-C₁₄)cycloalkylene group is replaced by a (C₃-C₁₄)aryl group. Examples of (C₃-C₁₄)aryl-(C₁-C₁₄)cycloalkylene groups include without limitation phenylcyclobutylene, phenylcyclopropylene and 3 -phenyl-2-methylbutylene-1-one.

The substituent —CO₂H, may be replaced with bioisosteric replacements such as:

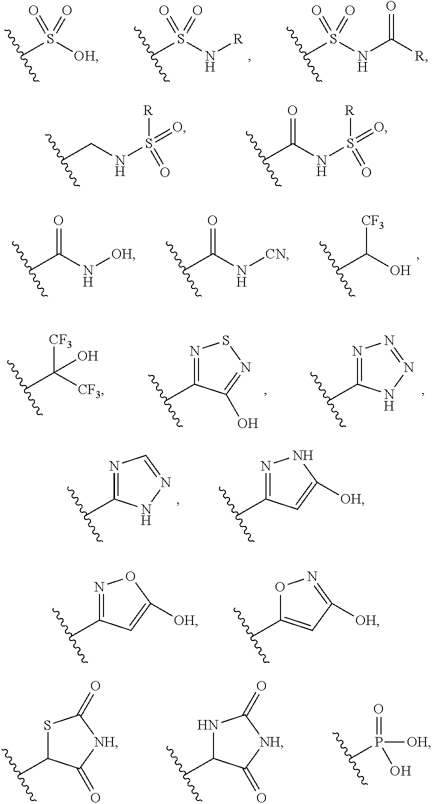

and the like, wherein R has the same definition as R′ and R″ as defined herein. See, e.g., THE PRACTICE OF MEDICINAL CHEMISTRY (Academic Press: New York, 1996), at page 203.

The compound of the invention can exist in various isomeric forms, including configurational, geometric, and conformational isomers, including, for example, cis- or trans-conformations. Compounds of the present invention may also exist in one or more tautomeric forms, including both single tautomers and mixtures of tautomers. The term “isomer” is intended to encompass all isomeric forms of a compound of this invention, including tautomeric forms of the compound. All forms are included in the invention.

Some compounds described here can have asymmetric centers and therefore exist in different enantiomeric and diastereomeric forms. A compound of the invention can be in the form of an optical isomer or a diastereomer. Accordingly, the invention encompasses compounds of the invention and their uses as described herein in the form of their optical isomers, diastereoisomers and mixtures thereof, including a racemic mixture. Optical isomers of the compounds of the invention can be obtained by known techniques such as asymmetric synthesis, chiral chromatography, simulated moving bed technology or via chemical separation of stereoisomers through the employment of optically active resolving agents.

Unless otherwise indicated, “stereoisomer” means one stereoisomer of a compound that is substantially free of other stereoisomers of that compound. Thus, a stereomerically pure compound having one chiral center will be substantially free of the opposite enantiomer of the compound. A stereomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, for example greater than about 90% by weight of one stereoisomer of the compound and less than about 10% by weight of the other stereoisomers of the compound, or greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound.

If there is a discrepancy between a depicted structure and a name given to that structure, then the depicted structure controls. Additionally, if the stereochemistry of a structure or a portion of a structure is not indicated with, for example, bold or dashed lines, the structure or portion of the structure is to be interpreted as encompassing all stereoisomers of it. In some cases, however, where more than one chiral center exists, the structures and names may be represented as single enantiomers to help describe the relative stereochemistry. Those skilled in the art of organic synthesis will know if the compounds are prepared as single enantiomers from the methods used to prepare them.

In this description, a “pharmaceutically acceptable salt” is a pharmaceutically acceptable, organic or inorganic acid or base salt of a compound of the invention. Representative pharmaceutically acceptable salts include, e.g., alkali metal salts, alkali earth salts, ammonium salts, water-soluble and water-insoluble salts, such as the acetate, amsonate (4,4-diaminostilbene-2,2-disulfonate), benzenesulfonate, benzonate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fiunarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosaliculate, suramate, tannate, tartrate, teoclate, tosylate, triethiodide, and valerate salts. A pharmaceutically acceptable salt can have more than one charged atom in its structure. In this instance the pharmaceutically acceptable salt can have multiple counterions. Thus, a pharmaceutically acceptable salt can have one or more charged atoms and/or one or more counterions.

The terms “treat”, “treating” and “treatment” refer to the amelioration or eradication of a disease or symptoms associated with a disease. In certain embodiments, such terms refer to minimizing the spread or worsening of the disease resulting from the administration of one or more prophylactic or therapeutic agents to a patient with such a disease.

The terms “prevent,” “preventing,” and “prevention” refer to the prevention of the onset, recurrence, or spread of the disease in a patient resulting from the administration of a prophylactic or therapeutic agent.

The term “effective amount” refers to an amount of a compound of the invention or other active ingredient sufficient to provide a therapeutic or prophylactic benefit in the treatment or prevention of a disease or to delay or minimize symptoms associated with a disease. Further, a therapeutically effective amount with respect to a compound of the invention means that amount of therapeutic agent alone, or in combination with other therapies, that provides a therapeutic benefit in the treatment or prevention of a disease. Used in connection with a compound of the invention, the term can encompass an amount that improves overall therapy, reduces or avoids symptoms or causes of disease, or enhances the therapeutic efficacy of or synergies with another therapeutic agent.

The terms “modulate”, “modulation” and the like refer to the ability of a compound to increase or decrease the function, or activity of, for example, the complex formed between cyclin-dependent kinases (cdk's) and their respective catalytic cyclin subunits. “Modulation”, in its various forms, is intended to encompass inhibition, antagonism, partial antagonism, activation, agonism and/or partial agonism of the activity associated with a cdk-cyclin complex. The ability of a compound to modulate the activity of cdk-cyclin complex can be demonstrated in an enzymatic assay or a cell-based assay.

A “patient” or subject” includes an animal, such as a human, cow, horse, sheep, lamb, pig, chicken, turkey, quail, cat, dog, mouse, rat, rabbit or guinea pig. The animal can be a mammal such as a non-primate and a primate (e.g., monkey and human). In one embodiment, a patient is a human, such as a human infant, child, adolescent or adult.

The term “prodrug” refers to a precursor of a drug, that is a compound which upon administration to a patient, must undergo chemical conversion by metabolic processes before becoming an active pharmacological agent. Exemplary prodrugs of compounds in accordance with Formula I are esters, dioxaborolanes, and amides.

Molecular Mechanism Associated with Inhibition of Viral Replication

The present invention implicates a role for viral microRNA (generated from full-length, doubly spliced or singly spliced HIV-1 transcripts), in inhibiting viral replication, blocking translation of viral proteins and causing a remodeling of the viral genome. Support for this hypothesis stems from the observation that association of integrated HIV-1 genome with chromatin remodeling complexes, and the role of histone acetyl-transferases activity in activation of the virus, indicates that viral microRNA machinery may have a significant role in controlling viral transcription.

The TAR element is a 50 nucleotide long sequence found at the 5′ end of the HIV-1 viral mRNA. TAR is one of the five structures within HIV that can serve as a substrate for Dicer. While the TAR element is involved in activating the promoter proximal region (PPR) of the HIV-1 gene and is involved only to a lesser extent with elongation of transcription, TAR according to this inventor plays an important role in regulating transcription and viral inhibition.

For instance, TAR hairpins are loaded into microRNA machinery by TAR-RNA Binding Protein (TRBP), identified as the human homologue of the Drosophila loquacious protein which is required for efficient loading of the microRNA into the RISC complex The fact that RNAi components, such as TRBP, can be found associated with the TAR element is strong evidence that TAR may be processed to yield microRNA.

The inventor hypothesizes without being bound to a particular theory that the production of TAR microRNA and binding to complementary genes could explain the down regulation of many cellular genes. Moreover, the observation that latent cells show a higher amount of short, abortive RNA transcripts (between 50-100 nucleotides in length), that contain the HIV-1 TAR hairpin, implicates that the microRNAs generated from TAR may act to suppress viral gene expression and alter host-cell proteins levels in order to maintain the latent state.

While current anti-retroviral drugs can halt viral replication, latent HIV-1 infections persist in infected patients. Any halt or interruption to the therapy quickly results in a resurgence of viral titers due to the reservoir of latent infections. Since HIV-1 TAR microRNA plays an important role in manipulating both cellular and viral mechanisms, it is not surprising that this viral microRNA could also be involved with drug efficacy.

As previously reported, cdk/cyclin complexes play an important role in viral replication. Effective cdk inhibitors are implicated, therefore, to play a significant role in suppressing viral replication and are candidate therapeutics for treating HIV-1 infection. For example, cdk inhibitor Cyc202 (R-Roscovitine), prevents cdk2/cyclin E binding to the HIV-1 LTR and inhibits HIV-1 in T-cells, monocytes, and peripheral blood mononuclear cells at a low IC50 (Agbottah et al, 2005).

Crystallographic data of the Cyc202 or its analog CR8 complexed to cdk show that the purine ring of Cyc202 and of CR8, are sandwiched between the side chain of Ile10 and of Leu134 of cdk2.

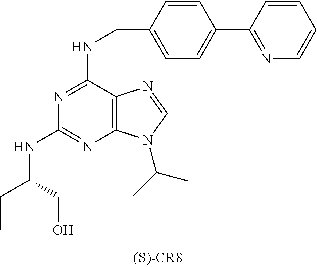

Compared to the Cyc202 phenyl group, the phenyl ring of CR8 is positioned further away from Phe82 side chain. Viral inhibition data suggested that CR8 is roughly 25 times more potent than Cyc202, while and initial drug screens by the inventor showed CR8 to effectively eliminate HIV-1 infected cells better than the uninfected cells.

CR8 was used as a lead compound, therefore, to synthesize analogs that are more potent inhibitors of viral replication. Based on data shown below, one of the most potent CR8 derivative was CR8#13, which significantly decreased viral transcription. Not only did CR8#13 down regulate viral transcription, it also did not affect cell viability or downstream cdk9 effector genes suggesting that CR8#13 is capable of specifically targeting HIV-1 transcripts.

### Compounds, Pharmaceutical Compositions and Methods of Use

As stated above the present invention is directed to providing Formula I compounds, their pharmaceutical compositions and methodologies for using the Formula I compounds or their pharmaceutical compositions to modulate HIV-1 transcription. HIV-1 produces several microRNAs including one from the TAR element which alter the host's response to infection. The host cell cycle is dependent on the activity of cyclin-dependent kinases (cdks) and their catalytic cyclin subunits for normal cell division and maturation activities. The cdk/cyclin complexes aid in the advancement of eukaryotic cell through the G1/S and G2/M cell cycle checkpoints. Since cyclin/cdk complexes are important for viral transcription, these studies focus on the possible cdk inhibitors that inhibit viral transcription, without affecting normal cellular mechanisms.

HIV-1 has the ability to manipulate the cdk/cyclin mechanisms within a cell to support its own life cycle. For example, HIV-1 targets the cdk2/cyclin E complex to allow cells to pass through the Gl/S checkpoint, enabling transcription of integral proliferative genes and to increase HIV-1 genome replication. The cdk/cyclin complexes are also linked to the viral proteins through their interaction with the HIV-1 Tat (trans activator of transcription) protein.

Tat is the main transcriptional activator of the HIV-1 LTR but also induces some cellular genes that help maintain viral production and/or cell survival (Bohan et al, 1992; Zhou et al, 2000). Tat binds the viral TAR element, and the Tat-TAR complex recruits viral and cellular components to initiate and elongate the viral promoter. For example, Tat recruits the pTEFb elongation complex to the promoter. The activated components of this complex, cdk9 and cyclin T1, then hyper-phosphorylate the large subunit of the RNA polymerase II C-terminal domain and other factors to activate transcription elongation (Kim et al, 2002). Therefore, inhibitors cdk/cyclin are candidate therapeutics for treatig HIV-1 infections.

According to an embodiment therefore, a Formula I compound, or a pharmaceutically acceptable salt, stereoisomer, tautomer, or prodrug thereof can be used to inhibit the replication of human immunodeficiency virus (HIV).

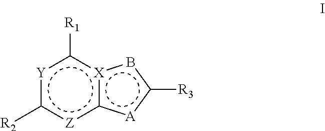

Without being bound to a specific theory, the present inventors believe that Formula I compounds similar to the cdk inhibitors Roscovitine and Flavopiridol, inhibit the activity of cdk1, 2, 5, 7, 9. Similar to Roscovitine and Flavopiridol, compounds conforming to Formula I are are highly potent suppressors of viral gene expression, rather than gene suppression in host cells at a drug concentration in the nanomolar range.

Accordingly, the present invention provides a process for inhibiting the replication of human immunodeficiency virus (HIV) by contacting an infected cell with at least one compound according to Formula I, or a pharmaceutical composition of a Formula I compound. For Formula I compounds, R₁ and R₂ are each independently selected from the group consisting of —H, straight or branched chain (C₁-C₆)alkyl, straight or branched chain (C₁-C₆)hydroxyalkyl, (C₂-C₆)alkene, (C₃-C₈)cycloalkyl, (C₃-C₁₄)aryl, halogen, —NR^{a}R^{b}, —NR^{a}(C₁-C₆)hydroxyalkyl, —NR^{a}(C₃-C₁₄)aryl(C₁-C₆)alkylene-, —NR^{a}(C₃-C₁₄)heteroaryl(C₁-C₆)alkylene-, —NR^{a}(C₃-C₁₄)heterocycloalkyl(C₁-C₆)alkylene-, —NR^{a}(C₃-C₁₄)cycloalkyl(C₁-C₆)alkylene-, —OR^{c}, and —SR^{c}.

According to Formula I, R₃ is selected from the group consisting of —H, straight or branched chain (C₁-C₆)alkyl, —OH and halogen and each of X, Y, Z, A and B is independently selected from the group consisting of a bond, —C(R′″)₂—, —CR′″—, —NR′″—, —N—, —O—, —C(O)—, and —S— and R′″ is defined below.

For Formula I compounds of the inventive pharmaceutical composition no more than three of X, Y, Z, A and B can simultaneously represent a bond; and X and B cannot simultaneously be —O—, or —S—. To accommodate for the presence of aromatic and non-aromatic ring systems, moreover, Formula I recites [symbol] to represent the option of having one or more double bonds.

For compounds encompassed by Formula I, R^{a}, R^{b}, R^{c} and R′″ are each independently selected from the group consisting of H, OH, straight or branched chain (C₁-C₈)alkyl, (C₃-C₆)aryl, —NH₂, —C(O)(C₁-C₆)alkyl, —C(O)(C₃-C₁₄)aryl, (C₃-C₆)cycloalkyl, (C₃-C₁₄)heterocycloalkyl, (C₃-C₁₄)heteroaryl, (C₃-C₁₄)aryl-(C₁-C₆)alkylene-, (C₃-C₆)cycloalkyl-(C₁-C₆)alkylene-, (C₃-C₁₄)heteroaryl-(C₁-C₆)alkylene-, (C₃-C₁₄)heterocycloalkyl-(C₁-C₆)alkylene-.

Furthermore, for compounds that conform to Formula I, any alkyl, alkylene, alkene, aryl, heteroaryl, cycloalkyl, or heterocycloalkyl is optionally substituted with one or more members selected from the group consisting of halogen, oxo, —COOH, —CN, —NO₂, —OH, straight or branched chain (C₁-C₆)alkyl, (C₂-C₆)alkene, (C₃-C₁₄)aryl, (C₃-C₁₄)heteroaryl, (C₃-C₁₄)heterocycloalkyl, (C₃-C₁₄)cycloalkyl, (C₁-C₆)alkoxy, and (C₃-C₁₄)aryloxy.

According to another embodiment, the invention provides a method for treating or preventing HIV infection in a subject by administering to the subject a therapeutically effective amount of at least one compound according to Formula I, or a pharmaceutically acceptable salt, stereoisomer, tautomer, or prodrug thereof.

In yet another embodiment is provides a method for treating human immunodeficiency virus (HIV) infection by administering to the subject a therapeutically effective amount of a viral transcription modulator, or a pharmaceutically acceptable salt, or prodrug thereof According to this method, compounds modulating viral transcription conform to Formula II.

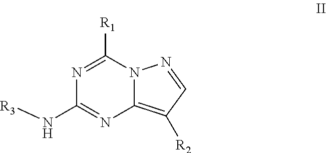

For Formula II compounds, R₁, R₂ and R₃ are each independently hydrocarbons. Within the context of the present invention the term “hydrocarbon” refers to any compound consisting of carbon and hydrogen atoms. Exemplary of such hydrocarbon include without limitation (C₁-C₆)alkyl, (C₂-C₆)alkenes, (C₂-C₆)alkynes, (C₃-C₈)cycloalkyl and (C₃-C₈)cycloalkenes.

For certain other Formula II compounds, however, R₁ is —NR^{a}(C₃-C₁₄)aryl(C₁-C₆)alkylene and R₂ and R₃ are each independently an optionally substituted (C₁-C₆)alkyl group. For Formula II compounds R^{a} is hydrogen, or a (C₁-C₆)alkyl group and any alkyl, aryl in Formula II is optionally substituted with one or more members selected from the group consisting of halogen, oxo, —COOH, —CN, —NO₂, —OH, straight or branched chain (C₁-C₆)alkyl, (C₂-C₆)alkene, (C₃-C₁₄)aryl, (C₃-C₁₄)heteroaryl, (C₃-C₁₄)heterocycloalkyl, (C₃-C₁₄)cycloalkyl, (C₁-C₆)alkoxy, and (C₃-C₁₄)aryloxy.

Exemplary of Formula I and Formula II compounds or their pharmaceutically acceptable salts used in inventive methodologies aimed at inhibiting the replication of HIV, or the treatment and/or prevention of HIV infection include without limitation those shown below in Table 1.

TABLE 1

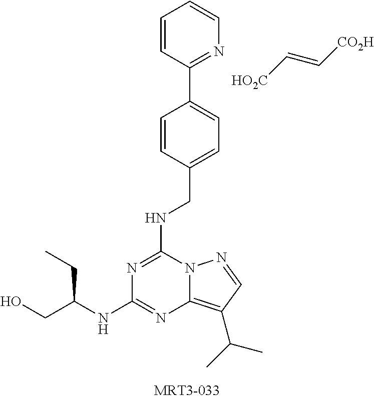

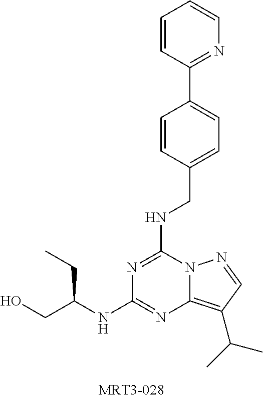

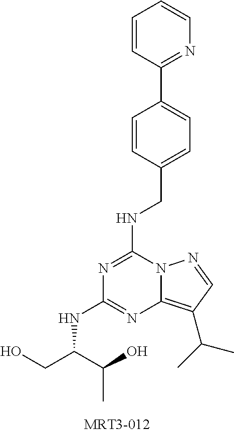

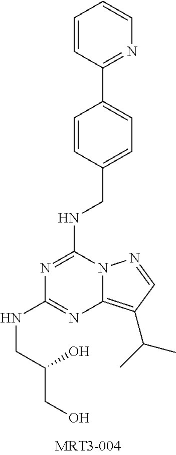

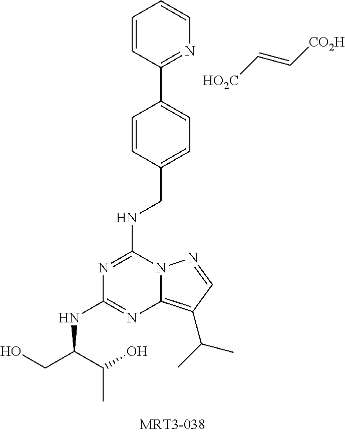

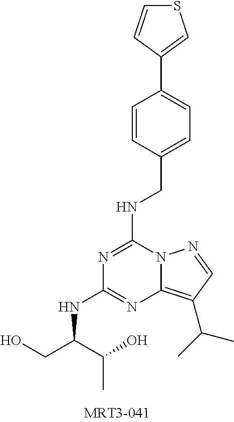

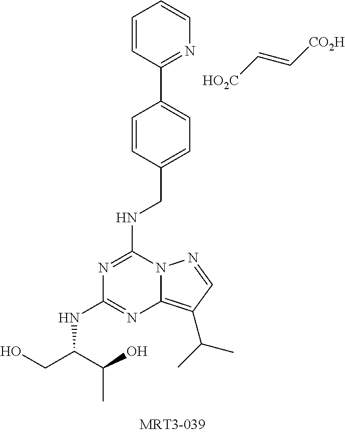

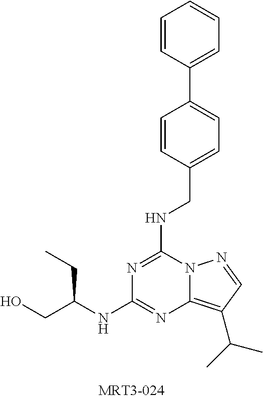

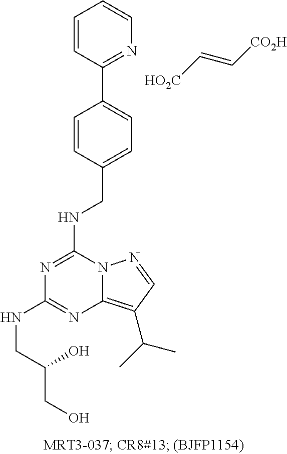

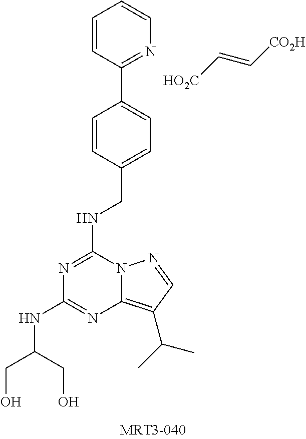

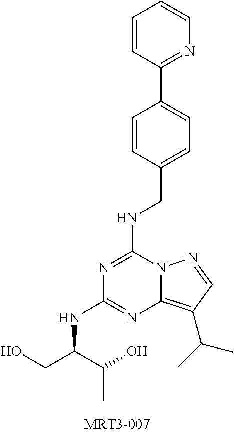

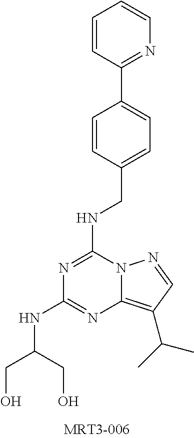

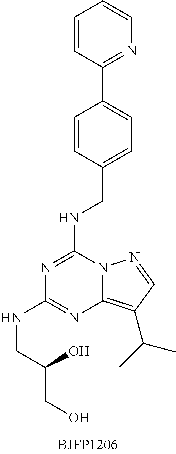

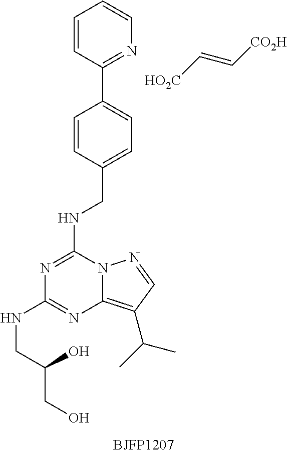

### B. Pharmaceutical Compositions and Dosages

Compounds according to Formula I, or Formula II are administered to a patient or subject in need of treatment either alone or in combination with other compounds having similar or different biological activities. For example, the compounds and compositions of the invention may be administered in a combination therapy, i.e., either simultaneously in single or separate dosage forms or in separate dosage forms within hours or days of each other. Examples of such combination therapies include administering the compositions and compounds of Formula I with other agents used to treat or prevent HIV infections.

Alternatively, compounds and compositions of the invention may be used to inhibit the replication of human immunodeficiency virus. Either single or multiple daily doses of a Formula I compound, or its compositions can be administered by a practicing medical practitioner.

Thus, in an embodiment, the invention provides a pharmaceutical composition comprising one or more compounds according to Formula I or a pharmaceutically acceptable salt, solvate, stereoisomer, tautomer, or prodrug, in admixture with a pharmaceutically acceptable carrier. In some embodiments, the composition further contains, in accordance with accepted practices of pharmaceutical compounding, one or more additional therapeutic agents, pharmaceutically acceptable excipients, diluents, adjuvants, stabilizers, emulsifiers, preservatives, colorants, buffers, flavor imparting agents.

In one embodiment, the pharmaceutical composition comprises a compound selected from those illustrated in Table 1 or a pharmaceutically acceptable salt, solvate, stereoisomer, tautomer, or prodrug thereof, and a pharmaceutically acceptable carrier.

The inventive compositions can be administered orally, topically, parenterally, by inhalation or spray or rectally in dosage unit formulations. The term parenteral as used herein includes subcutaneous injections, intravenous, intramuscular, intrasternal injection or infusion techniques.

Suitable oral compositions in accordance with the invention include without limitation tablets, troches, lozenges, aqueous or oily suspensions, dispersible powders or granules, emulsion, hard or soft capsules, syrups or elixirs.

Encompassed within the scope of the invention are pharmaceutical compositions suitable for single unit dosages that comprise a compound of the invention its pharmaceutically acceptable stereoisomer, prodrug, salt, solvate, hydrate, or tautomer and a pharmaceutically acceptable carrier. Typical systemic dosages for treating or preventing HIV replication, or for inhibiting viral replication include doses ranging from 0.01 mg/kg to 1500 mg/kg of body weight per day as a single daily dose or divided daily doses. Preferred dosages for the described conditions range from 0.5-1500 mg per day. A more particularly preferred dosage for the desired conditions ranges from 5-750 mg per day. Typical dosages can also range from 0.01 to 1500, 0.02 to 1000, 0.2 to 500, 0.02 to 200, 0.05 to 100, 0.05 to 50, 0.075 to 50, 0.1 to 50, 0.5 to 50, 1 to 50, 2 to 50, 5 to 50, 10 to 50, 25 to 50, 25 to 75, 25 to 100, 100 to 150, or 150 or more mg/kg/day, as a single daily dose or divided daily doses. In one embodiment, the compounds are given in doses of between about 1 to about 5, about 5 to about 10, about 10 to about 25 or about 25 to about 50 mg/kg.

Inventive compositions suitable for oral use may be prepared according to any method known to the art for the manufacture of pharmaceutical compositions. For instance, liquid formulations of the inventive compounds contain one or more agents selected from the group consisting of sweetening agents, flavoring agents, coloring agents and preserving agents in order to provide pharmaceutically elegant and palatable preparations of the inventive cdk inhibitor.

For tablet compositions, the active ingredient in admixture with non-toxic pharmaceutically acceptable excipients is used for the manufacture of tablets. Exemplary of such excipients include without limitation inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known coating techniques to delay disintegration and absorption in the gastrointestinal tract and thereby to provide a sustained therapeutic action over a desired time period. For example, a time delay material such as glyceryl monostearate or glyceryl distearate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

For aqueous suspensions the inventive compound is admixed with excipients suitable for maintaining a stable suspension. Examples of such excipients include without limitation are sodium carboxymethylcellulose, methylcellulose, hydropropylmethylcellulose, sodium alginate, polyvinylpyrrolidone, gum tragacanth and gum acacia.

Oral suspensions can also contain dispersing or wetting agents, such as naturally-occurring phosphatide, for example, lecithin, or condensaturatedion products of an alkylene oxide with fatty acids, for example polyoxyethylene stearate, or condensaturatedion products of ethylene oxide with long chain aliphatic alcohols, for example, heptadecaethyleneoxycetanol, or condensaturatedion products of ethylene oxide with partial esters derived from fatty acids and a hexitol such as polyoxyethylene sorbitol monooleate, or condensaturatedion products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions may be formulated by suspending the active ingredients in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol.

Sweetening agents such as those set forth above, and flavoring agents may be added to provide palatable oral preparations. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

Compositions for parenteral administrations are administered in a sterile medium. Depending on the vehicle used and concentration the concentration of the drug in the formulation, the parenteral formulation can either be a suspension or a solution containing dissolved drug. Adjuvants such as local anesthetics, preservatives and buffering agents can also be added to parenteral compositions.

### Synthesis of Compounds

Compounds of the invention are prepared using general synthetic methods disclosed in PCT publication No. WO 2010/103486 the subject matter of which is incorporated in its entirety by reference and further described below. The choice of an appropriate synthetic methodology is guided by the choice of Formula I compound desired and the nature of functional groups present in the intermediate and final product. Thus, selective protection/deprotection protocols may be necessary during synthesis depending on the specific functional groups desired and protecting groups being used. A description of such protecting groups and how to introduce and remove them is found in PROTECTIVE GROUPS IN ORGANIC SYNTHESIS (3^{rd} ed.), T. W. Green and P. G. M. Wuts, John Wiley and Sons, New York (1999).

Exemplary general synthetic methodologies for making Formula I compounds are provided below. More specific syntheses of illustrative Formula I compounds are also provided.

Briefly Formula I compounds having a pyrazolo-trazine core can readily be synthesized as shown in Scheme 1 below:

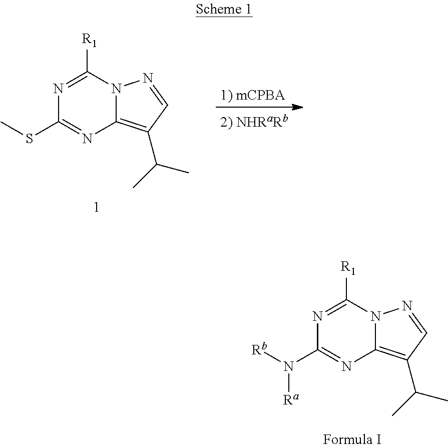

According to Scheme 1, oxidation of the thioether using meta-chloroperbenzoic acid (mCPBA), in dichloromethane at 0° C., gave the corresponding sulfone which is used directly in an aromatic nucleophilic substitution reaction (SN_{AR}) in the presence of a primary amine at a temperature ranging from 100 to 180° C. , to yield the compound a Formula (I).

Compound (1) (Scheme 1) is prepared by a palladium (Miyaura-Suzuki reaction or Stille) or tin catalyzed cross-coupling reaction between an aryl halide and a appropriate boronate or stanate as shown in Scheme 2

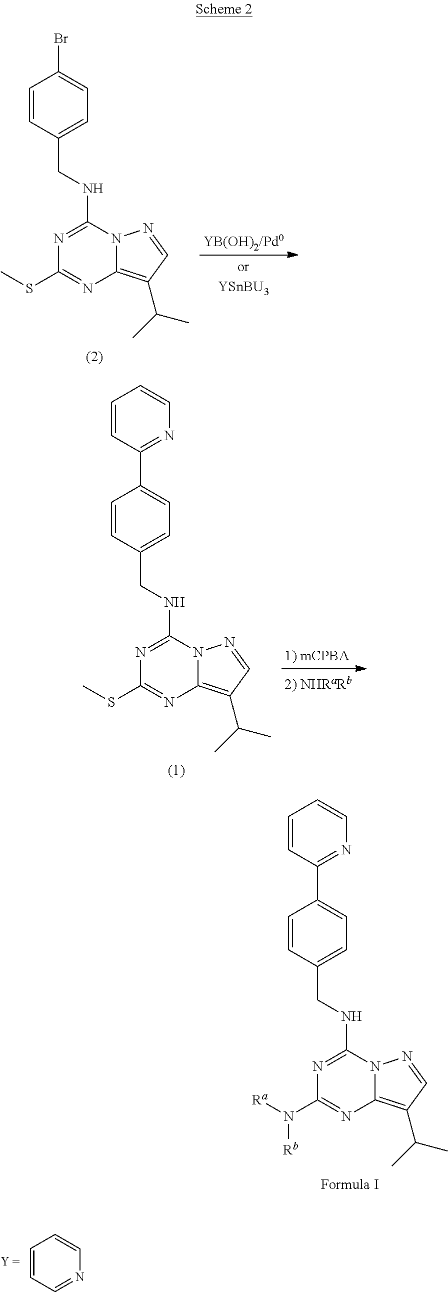

Compound (1) was oxidized in the presence of meta-chloroperbenzoic acid in dichloromethane at 0° C. to yield the sulfone which underwent a nucleophilic substitution reaction with a primary amine to give a Formula I compound.

Synthesis of R-2-(4-(biphenyl-4-ylmethylamino)-8-isopropyl-pyrazolo[1,5-α]-1,3,5-triazine-2-ylaminobutan-1-ol (MRT3-024)

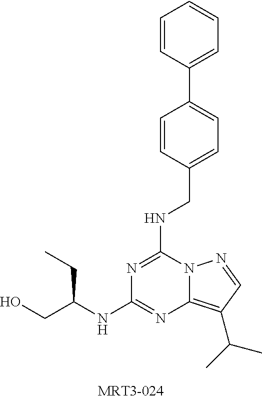

A solution of N-(biphenyl-4-ylmethyl)-8-isopropyl-2-(methylthio)pyrazolo[1,5-α]-1,3,5-triazine-4-amine in CH₂Cl₂ (4 mL) was stirred at 0° C. Meta-chloroperbenzoic acid 70-75% (100 mg, 0.41 mmol) was added and the solution was stirred for 1 h. The same amount of mCPBA is added again. The final reaction mixture was stirred for 2 h at 0° C. The reaction was quenched using an aqueous solution of NaHCO₃, and the reaction mixture partitioned to separate the organic and aqueous phases. The organic phase is washed with more bicarbonate and water. The isolated organic phase is finally washed with NaCl solution, dried over MgSO₄ and evaporated under reduced pressure. The desired sulfone is obtained in quantitative yield and used without further purification.

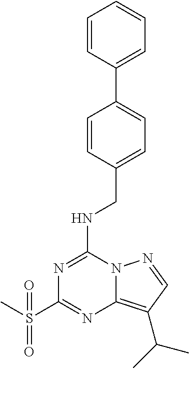

A solution of N-(biphenyl-4-ylmethyl)-8-isopropyl-2-(methylsulfonyl)pyrazolo[1,5-α]-1,3,5-triazin-4-amine (173 mg, 0.41 mmol) and (R)-(—)-2-aminobutanol commercial (193 microL, 2.03 mmol) were heated to 140° C. for 24 h. After cooling, the solvent was evaporated. The crude product was purified by flash chromatography (petroleum ether/AcOEt 8:2 and 1:1) to give the desired compound (75 mg, 43%). Oil. ¹H NMR (300 MHz, CDCl₃): δ 7.63 (s, IH, Hₐᵣₒₘ) 7.59 to 7.56 (m, 4H, Hₐᵣₒₘ) 7.47 to 7.33 (m, 5H, Hₐᵣₒₘ), 6.74 (salt, 1H, NH), 4.78 to 4.75 (m, 2H, CH₂), 3.94 to 3.96 (salt, 1H, CH), 3, 82 (d, IH, J=10.8 Hz, CH₂), 3.68 (dd, IH, J=7.3, 10.8 Hz, CH₂), 3.02 (hept, IH, J=6.6 Hz, CH), 1.70 to 1.52 (m, 2H, CH₂), 1.28 (d, 6H, J=6.8 Hz, 2CH₃), 1.03 (t, 3H, J=7.4 Hz, CH₃). MS (ESI): m/z 431 (MH).

Synthesis of R-2-(8-isopropyl-4-(4-(pyridine-2-yl)benzylamino)pyrazolo[1,5-α]-1,3,5-triazine-2-ylamino)butan-1-ol (MRT3-028)

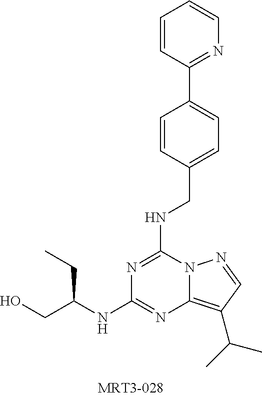

This compound was synthesized using a protocol similar to that described above for MRT3-024^{. 1}H NMR (300 MHz, CDCl₃): δ 8.69 (d, IH, J=4.1 Hz, Hₐᵣₒₘ), 7.96 (d, 2H, J=7.9 Hz, Hₐᵣₒₘ), 7.78 to 7.69 (m, 2H, Hₐᵣₒₘ), 7.62 (s, 1H, Hₐᵣₒₘ), 7.42 (d, 2H, J=7.9 Hz, Hₐᵣₒₘ), 7.026 to 7.21 (m, 1H, Hₐᵣₒₘ), 6.91 (salt, IH, NH), 5.10 (s, 1H, exchangeable H), 4.75 (d, 2H, J=6.0 Hz, CH₂), 3.93 (salt, 1H, CH), 3.83 to 3.62 (m, 2H, CH₂), 3.02 (hept, 1H, J=6.8 Hz, CH), 1.70 to 1.52 (m, 2H, CH₂), 1.27 (d, 6H, J=6.8 Hz, 2CH₃), 1.02 (t, 3H, J=7.4 Hz, CH₃). MS (ESI): m/z 432 (MH⁺).

Synthesis of the fumarate salt of R-2-(8-isopropyl-4-(4-(pyridine-2-yl)benzylamino)pyrazolo[1,5-α]-1,3,5-triazine-2-ylamino)butan-1-ol (MRT3-033)

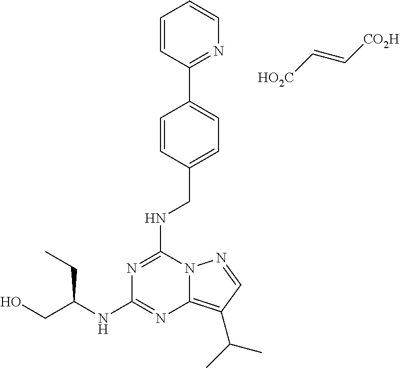

MRT3-028 was treated with fumaric acid using a mixture of EtOH/Et₂O as the solvent. The desired fumarate salt crystallizes at 0° C. Mp 175-177° C. ¹H NMR (300 MHz, DMSO-J₆): δ 13.12 (salt, 2H, OH), 8.70 (salt, IH, NH), 8.64 (d, IH, J=4.1 Hz, Hₐᵣₒₘ), 8.03 (d, 2H, J=8.3 Hz, Hₐᵣₒₘ), 7.92 (d, IH, J=8.0 Hz, Hₐᵣₒₘ), 7.86 (t, 1H, J=8.0 Hz, Hₐᵣₒₘ), 7.70 (s, 1H, Hₐᵣₒₘ), 7.48 (d, 2H, J=8.3 Hz, Hₐᵣₒₘ), 7.35 to 7.31 (m, IH, Hₐᵣₒₘ), 6.62 (s, 2H, ═CH), 6.51 (salt, 1H, NH), 4.67 (salt, 2H, CH₂), 4.51 (salt, IH, OH), 3.82 (salt, 1H, CH), 3.45 to 3.32 (m, 2H, CH₂), 2.90 (hept, 1H, J=6.8 Hz, CH), 1.65 to 1.35 (m, 2H, CH₂), 1.23 (d, 6H, J=6.8 Hz, 2CH₃), 0.84 (t , 3H, J=7.4 Hz, CH₃).

Synthesis of S-3-(8-isopropyl-4-(4-(pyridine-2-yl)benzylamino)pyrazolo[1,5-α]-1,3,5-triazine-2-ylamino)propoane-1,2-diol (MRT3-004)

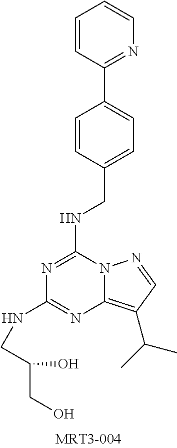

Synthesis of the target compound was achieved by reacting the sulfone 8-isopropyl-2-(methylsulfonyl)-N-(4-(pyridine-2-yl)benzylamino)pyrazolo[1,5-α]-1,3,5-triazine-4-amine with (S)-3-amino-1,2-propoane diol. Yield=20%. Oil ¹H NMR (300 MHz, CDCl₃): δ 8.69 (d, IH, J=4.5 Hz, Hₐᵣₒₘ), 7.98 (d, 2H, J=8.3 Hz, Hₐᵣₒₘ), 7.79 to 7.70 (m, 2H, Hₐᵣₒₘ), 7.66 (s, 1H, Hₐᵣₒₘ), 7.45 (d, 2H, J=8.3 Hz, Hₐᵣₒₘ), 7.27 -7.22 (m, 1H, Hₐᵣₒₘ), 6.81 (1H, NH), 4.78 (d, 2H, J=5.8 Hz, CH₂), 3.83 to 3.79 (m, IH, CH) , 3.65-3.55 (m, 4H, 2CH₂), 3.03 (hept, IH, J=7.0 Hz, CH), 1.28 (d, 6H, J=7.0 Hz, 2CH₃). MS (ESI): m/z 434 (MH⁺).

Synthesis of the fumarate salt of S-3-(8-isopropyl-4-(4-(pyridine-2-yl)benzylamino)pyrazolo[1,5-α]-1,3,5-triazine-2-ylamino)propoane-1,2-diol (MRT3-037)

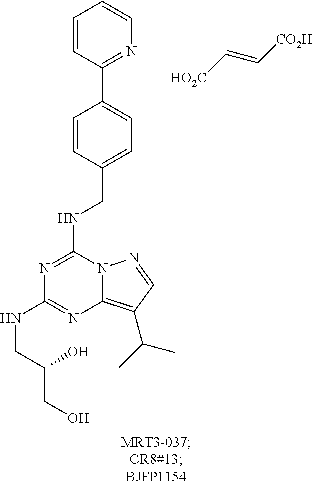

MRT3-004 was treated with fumaric acid using a mixture of EtOH/Et₂O as the solvent. The desired fumarate salt crystallizes at 0° C. Mp 168-170° C. ¹H NMR (300 MHz, CDCl₃+1 drop DMSO-J₆): δ 8.64 (d, 1H, J=3.8 Hz, Hₐᵣₒₘ), 7.93 (d, 2H, J=8.1Hz, Hₐᵣₒₘ), 7.74 to 7.65 (m, 2H, Hₐᵣₒₘ), 7.60 (s, 1H, Hₐᵣₒₘ), 7.42 (d, 2H, J=8.1 Hz, Hₐᵣₒₘ), 7.26 to 7.18 (m, IH, Hₐᵣₒₘ), 6.76 (s, 2H, ═CH), 4.74 (d, 2H, J=5.9 Hz, CH₂), 3.80 to 3.75 (m, 1H, CH), 3.56 to 3.45 (m, 4H, 2CH₂), 2.99 (hept, 1H, J=6.8 Hz, CH), 1.23 (d, 6H, J=6.8 Hz, 2 CH₃).

Synthesis of R-3-(8-isopropyl-4-(4-(pyridine-2-yl)benzylamino)pyrazolo[1,5-α]-1,3,5-triazine-2-ylamino)propoane-1,2-diol (BJFP1206)

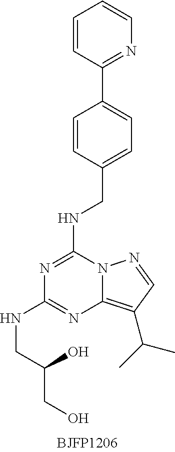

Synthesis of the target compound was achieved by reacting the sulfone 8-isopropyl-2-(methylsulfonyl)-N-(4-(pyridine-2-yl)benzylamino)pyrazolo[1,5-α]-1,3,5-triazine-4-amine with (R)-3-amino-1,2-propoane diol. Yield=35%. Oil. ¹H NMR (300 MHz, CDCl₃): δ 8.69 (d, 1H, J=4.5 Hz, Hₐᵣₒₘ), 7.98 (d, 2H, J=8.3 Hz, Hₐᵣₒₘ), 7.79 to 7.70 (m, 2H, Hₐᵣₒₘ), 7.66 (s, 1H, Hₐᵣₒₘ), 7.45 (d, 2H, J=8.3 Hz, Hₐᵣₒₘ), 7.27 0.28 to 7.22 (m, 1H, Hₐᵣₒₘ), 6.80 (sél, H, NH), 4.78 (d, 2H, J=5.8 Hz, CH₂), 3.83-3.79 (m, 1H, CH), 3.65-3-3.55 (m, 4H, 2CH₂), 3.03 (hept, 1H, J=7.0 Hz, CH), 1.28 (d, 6H, J=7.0 Hz, 2CH₃). MS (ESI): m/z 434 (MH⁺).

Synthesis of the fumarate salt of R-3-(8-isopropyl-4-(4-(pyridine-2-yl)benzylamino)pyrazolo[1,5-α]-1,3,5-triazine-2-ylamino)propoane-1,2-diol (BJFP1207)

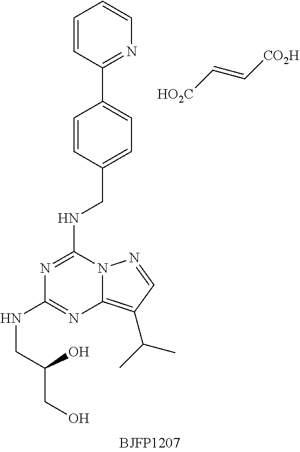

BJFP 1206 was treated with fumaric acid using a mixture of EtOH/Et₂O as the solvent. The desired fumarate salt crystallizes under cold conditions. Mp 168-170° C. ¹H NMR (300 MHz, CDCl₃+1 drop DMSO-d₆): δ 8.64 (d, IH, J=3.8 Hz, Hₐᵣₒₘ), 7.93 (d, 2H, J=8.1 Hz, Hₐᵣₒₘ), 7.74 to 7.65 (m, 2H, Hₐᵣₒₘ), 7.60 (s, IH, Hₐᵣₒₘ), 7.42 (d, 2H , J=8.1 Hz, Hₐᵣₒₘ), 7.26 to 7.18 (m, 1H, Hₐᵣₒₘ), 6.76 (s, 2H, ═CH), 4.74 (d, 2H, J=5.9 Hz, CH₂), 3.80 to 3.75 (m, 1H, CH), 3.56 to 3.45 (m, 4H, 2CH₂), 2.99 (hept, 1H, J=6.8 Hz, CH), 1.23 (d, 6H, J=6.8 Hz, 2CH₃).

Synthesis of (2S, 3S)-2-(8-isopropyl-4-(4-(pyridine-2-yl)benzylamino)pyrazolo[1,5-α]-1,3,5-triazine-2-ylamino)butane-1,3-diol (MRT3-012)

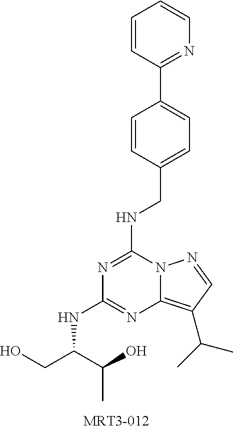

Synthesis of the target compound was achieved by reacting the sulfone 8-isopropyl-2-(methylsulfonyl)-N-(4-(pyridine-2-yl)benzylamino)pyrazolo[1,5-α]-1,3,5-triazine-4-amine with L-threoninol ((2S, 3S)-2-aminobutane-1,3-diol). Yield=43%. Oil. ¹H NMR (300 MHz, CDCl₃): δ 8.69 (d, 1IH, J=4.7 Hz, Hₐᵣₒₘ), 7.97 (d, 2H, J=8.3 Hz, Hₐᵣₒₘ), 7.78 to 7.69 (m, 2H, Hₐᵣₒₘ), 7.63 (s, 1H, Hₐᵣₒₘ), 7.45 (d, 2H, J=8.3 Hz, Hₐᵣₒₘ), 7.26-7.21 (m, IH, Hₐᵣₒₘ), 6.75 (1H, NH), 5.63 (d, IH, J=5.8 Hz, NH), 4.77 (d, 2H, J=6.0 Hz, CH₂), 4.22 to 4.15 (m, 1H, CH), 3.97 to 3.84 (m, 3H, CH+CH₂), 3.01 hept, (1H, J=7.0 Hz, CH), 1.29 (d, 3H, J=6.2 Hz, CH₃), 1.28 (d, 6H, J=7.0 Hz, 2CH₃). SM (ESI): m/z 448 (MH⁺).

Synthesis of the fumarate salt of (2S, 3S)-2-(8-isopropyl-4-(4-(pyridine-2-yl)benzylamino)pyrazolo[1,5-α]-1,3,5-triazine-2-ylamino)butane-1,3-diol (MRT3-039)

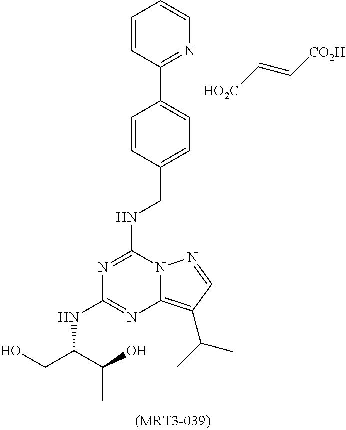

MRT3-012 was treated with fumaric acid using a mixture of EtOH/Et₂O as the solvent. The desired fumarate salt crystallizes under cold conditions. Mp 187-189° C. ¹H NMR (300 MHz, DMSO-d₆): δ 13.12 (salt, 2H, OH), 8.77 (salt, 1H, NH), 8.64 (d, 1H, J=4.5 Hz, Hₐᵣₒₘ), 8.03 (d, 2H, J=8.1 Hz, Hₐᵣₒₘ), 7.94 to 7.82 (m, 2H, Hₐᵣₒₘ), 7.72 (s, 1H, Hₐᵣₒₘ), 7.49 (salt, 2H, Hₐᵣₒₘ), 7.35-7.31 (m, IH, Hₐᵣₒₘ), 6.62 (s, 2H, ═CH2) 6.06 (d, IH, J=8.7 Hz, NH), 4.73 (salt, 2H, CH₂), 4.00 to 3.89 (salt, IH, CH), 3.89 to 3.76 (salt, IH, CH), 3.58 to 3.40 (m, 2H, CH₂), 2.90 (hept, IH, J=7.0 Hz, CH), 1.22 (d, 6H , J=5.8 Hz, 2CH₃), 1.04 (salt, 3 H CH₃).

Synthesis of (2R, 3R)-2-(8-isopropyl-4-(4-(pyridine-2-yl)benzylamino)pyrazolo[1,5-α]-1,3,5-triazine-2-ylamino)butane-1,3-diol (MRT3-007)

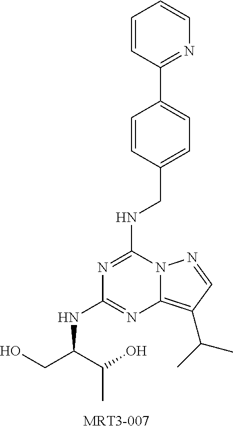

Synthesis of the target compound was achieved by reacting the sulfone 8-isopropyl-2-(methylsulfonyl)-N-(4-(pyridine-2-yl)benzylamino)pyrazolo[1,5-α]-1,3,5-triazine-4-amine with D-threoninol ((2R, 3R)-2-aminobutane-1,3-diol). Yield=42%. Oil. ¹H NMR (300 MHz, CDCl₃): δ 8.69 (d, 1H, J=4.7 Hz, Hₐᵣₒₘ), 7.97 (d, 2H, J=8.3 Hz, Hₐᵣₒₘ), 7.78 to 7.69 (m, 2H, Hₐᵣₒₘ), 7.63 (s, 1H, Hₐᵣₒₘ), 7.45 (d, 2H, J=8.3 Hz, Hₐᵣₒₘ), 7.26-7.21 (m, 1H, Hₐᵣₒₘ), 6.71 (Salt, 1H, NH), 5.62 (d, H, J=6.6 Hz, NH), 4.77 (d, 2H, J=6.0 Hz, CH₂), 4.22 to 4.15 (m, 1H, CH), 3.97 to 3.84 (m, 3H, CH+CH₂), 3.01 (hept , 1H, J=7.0 Hz, CH), 1.29 (d, 3H, J=6.2 Hz, CH₃), 1.28 (d, 6H, J=7.0 Hz, 2CH₃). SM (ESI): m/z 448 (MH⁺).

Synthesis of (2R, 3R)-2-(8-isopropyl-4-(4-(pyridine-2-yl)benzylamino)pyrazolo[1,5-α]-1,3,5-triazine-2-ylamino)butane-1,3-diol fumarate (MRT3-038)

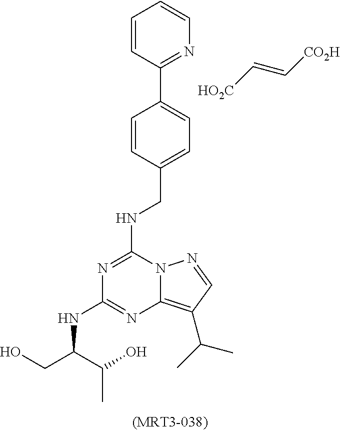

Mp 187-189° C. ¹H NMR (300 MHz, DMSO-d₆): δ 13.12 (salt, 2H, OH), 8.75 (salt, 1H, NH), 8.64 (d, IH, J=4.5 Hz, Hₐᵣₒₘ), 8.03 (d, 2H, J=8.1 Hz, Hₐᵣₒₘ), 7.94 to 7.82 (m, 2H, Hₐᵣₒₘ), 7.72 (s, 1H, Hₐᵣₒₘ), 7.49 (salt, 2H, Hₐᵣₒₘ), 7.35-7.31 (m, IH, Hₐᵣₒₘ), 6.62 (s, 2H, ═CH2) 6.05 (d, IH, J=8.7 Hz, NH), 4.67 (salt, 2H, CH₂), 4.00 to 3.89 (salt, 1H, CH), 3.89 to 3.76 (salt, 1H, CH), 3.58 to 3.40 (m, 2H, CH₂), 2.90 (hept, IH, J=7.0 Hz, CH), 1.22 (d, 6H, J=5.8 Hz, 2CH₃), 1.04 (salt, 3H₅CH₃).

Synthesis of (2R, 3R)-2-(8-isopropyl-4-(4-(pyridine-2-yl)benzylamino)pyrazolo[1,5-α]-1,3,5-triazine-2-ylamino)butane-1,3-diol fumarate (MRT3-006)

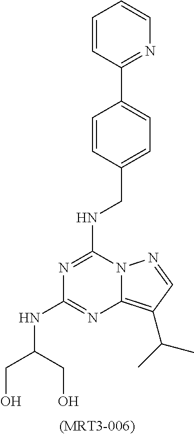

Synthesis of the target compound was achieved by reacting the sulfone 8-isopropyl-2-(methylsulfonyl)-N-(4-(pyridine-2-yl)benzylamino)pyrazolo[1,5-α]-1,3,5-triazine-4-amine with 2-aminopropane-1,3-diol. Yield=30%. Oil. ¹H NMR (300 MHz, CDCl₃): δ 8.68 (d, 1H, J=4.4 Hz, Hₐᵣₒₘ), 7.96 (d, 2H, J=8.3 Hz, Hₐᵣₒₘ), 7.79 to 7.69 (m, 2H, Hₐᵣₒₘ), 7.64 (s, 1H, Hₐᵣₒₘ), 7.45 (d, 2H, J=8.3 Hz, Hₐᵣₒₘ), 7 0.26 to 7.21 (m, 1H, Hₐᵣₒₘ), 6.80 (salt, 1H, NH), 5.71 (salt, IH, NH), 4.77 (d, 2H, J=5.9 Hz, CH₂), 4.08 to 4.95 (m, 1H, CH), 3.92 to 3.80 (m, 4H, 2CH₂), 3.02 (hept, IH, J=7.0 Hz, CH), 1.28 (d, 6H, J=7.0 Hz, 2CH₃). MS (ESI): m/z 434 (MH⁺).

Synthesis of the fumarate salt of (2R, 3R)-2-(8-isopropyl-4-(4-(pyridine-2-yl)benzylamino)pyrazolo[1,5-α]-1,3,5-triazine-2-ylamino)butane-1,3-diol fumarate(MRT3-040)

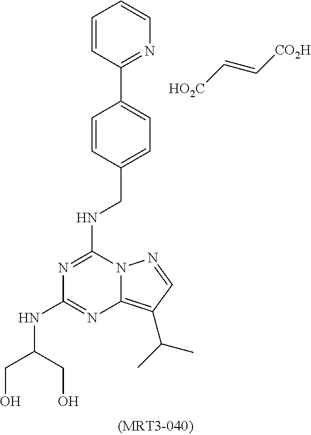

MRT3-006 was treated with fumaric acid using a mixture of EtOH/Et₂O as the solvent. The desired fumarate salt crystallizes under cold conditions Mp=179-181° C. ¹H NMR (300 MHz, DMSO-d₆): δ 13.11 (salt, 2H, OH), 8.76 (salt, IH, NH), 8.64 (d, 1H, J=4.5 Hz, Hₐᵣₒₘ), 8.03 (d, 2H, J=8.1 Hz, Hₐᵣₒₘ), 7.94 to 7.82 (m, 2H, Hₐᵣₒₘ), 7.72 (s, 1H, Hₐᵣₒₘ), 7.49 (salt, 2H, Hₐᵣₒₘ), 7.35-7.31 (m, 1H, Hₐᵣₒₘ), 6.63 (s, 2H, 2=CH) 6.33 (d, 1H, J=6.0 Hz, NH), 4.67 (d, 2H, J=6.0 Hz, CH₂), 3.98 to 3.83 (m, 1H, CH), 3.58 to 3.42 (m, 4H, 2CH₂), 2.91 (hept, 1H, J=7.0 Hz, CH), 1.23 (d, 6H, J=7.0 Hz, 2CH₃).

### METHODS AND USES

The inventive compounds are useful for treating and preventing HIV infection in a subject. Compounds according to Formula I are also potent inhibitors of viral replicationvia the inhibition of cdk-cyclin complex that plays an important role in viral transcription.

### 1. Cell Culture and Reagents

TZM-bl cell lines were grown in Dulbecco's modified Eagle's medium supplemented with fetal bovine serum (FBS) (10%, v/v), 2 mM L-glutamine, and antibiotics (penicillin 100 U/ml, streptomycin 100 mg/m1) (cDMEM). HCT116 WT and HCT116 Dicer−/−cell lines were grown in McCoy's medium supplemented with FBS (10%, v/v), 2mM L-glutamine, and antibiotics (penicillin 100 U/ml, streptomycin 100 mg/ml). CEM, ACH2, Jurkat and U937 cells were grown in RPMI 1640 supplemented with FBS, L-glutamine, and antibiotics (penicillin 100 U/ml, streptomycin 100 mg/ml). All cell lines were maintained at 37° C. in 5% (v/v) CO2.

ACH2 cells are infected with HIV-1; TZM-bl cells contain a stably-integrated HIV-1 LTR-Luciferase reporter; CEM, Jurkat, and U937 cells are uninfected. Transfections were carried out using Attractene (Qiagen) lipid reagent. Cells were cultured to confluence and pelleted at 4° C. for 15 min at 3,000 rpm. The cell pellets were washed twice with 25 ml of phosphate-buffered saline (PBS) with Ca2+ and Mg2+ (Quality Biological) and centrifuged once more. Cell pellets were resuspended in lysis buffer (50 mM Tris-HCl, pH 7.5, 120 mM NaCl, 5 mM EDTA, 0.5%, v/v, NP-40, 50 mM NaF, 0.2 mM Na3VO4, 1 mM DTT, one complete protease cocktail tablet/50 ml (Roche) and incubated on ice for 20 min, with gentle vortexing every 5 min. Cell lysates were transferred to eppendorf tubes and were centrifuged at 10,000 rpm for 10 min. Supernatants were transferred to a fresh tube where protein concentrations were determined using Bio-Rad protein assay (Bio-Rad, Hercules, Calif.).

### RT-PCR and Primers

For mRNA analysis of cdk9 related genes following drug treatments, total RNA was isolated from cells using Trizol (Invitrogen) according to the manufacturer's protocol. A total of 1 μg of RNA from the RNA fraction was treated with 0.25mg/ml DNase I for 60 min, followed by heat inactivation at 65° C. for 15 min. A total of 1 μg of total RNA was used to generate cDNA with the iScript cDNA Synthesis kit (Bio-Rad) using oligo-dT reverse primers.

### Electroporation and Reverse Transcriptase Assay

For electroporations, Jurkat and U937 cells were resuspended at 3 million cells in 250 μl of RPMI. Five microgram of pNL4-3 was then added to the cell suspension. Cells were pulsed a single time at 210V, 800 μF, and low resistance. Electroporated cells were immediately transferred to RPMI-1640 with L-glutamine and Penicillin/Streptomycin with 10% (v/v) FBS and plated in 6 well plates. Twenty four hours-post electroporation, cells were treated with drugs for additional 48 hrs and harvested for RT analysis. RT assays were performed as described in ((Guendel et al. 2009; Easley et al. 2010).

### Poly-A RT-PCR

For poly-A RT-PCR detection of microRNAs, 500 ng of RNA from the microRNA-enriched fraction was used to generate cDNA using the Quantimir kit (SBI) according to the manufacturer's protocol. Briefly, small RNA species are poly-adenylated and then reverse transcription reactions are performed with a company-provided RT primer. For PCR, a universal reverse primer is provided by the manufacturer. Specific microRNA forward primers are identical in sequence to the microRNA of interest. PCR products corresponding to the amplified microRNAs were separated in a 3.5% (w/v) agarose gel and quantified using the Kodak 1D software.

### Chloramphenicol Acetyltransferase (CAT) Assay

Various cells lines were transfected with the HIV-1 LTRCAT plasmid and pc-Tat to initiate the LTR transcription, and subsequently subjected to various drug treatments. After 48 hrs, cells were lysed and CAT assays were performed as previously described (Guendel et al, 2009; Van Duyne et al, 2008; Easley et al, 2010).

### Luciferase Assay

TZM-bl cells were transfected with pc-Tat (0.5 μg) using Attractene reagent (Qiagen) according to the manufacturers' instructions. Twenty-four hours later, cells were treated with DMSO or the indicated compound. Forty-eight hours-post drug treatment, luciferase activity of the firefly luciferase was measured with the BrightGlo Luciferase Assay (Promega).

### RNase Protection Assay (RPA)

Total RNA was extracted from TNF-treated CEM and ACH2 cells follow by drug treatment with Cyc202 (500 nm), CR8 (100 μM), CR8#13 (50 nm), and Flavopiridol (50 nm) for 48 hrs using Trizol reagent (Invitrogen). RNase protection assays were performed as previously described (Klase et al, 2007).

### Chromatin Immunoprecipitation Assay (ChIP)

TZM-bl cells were treated with TSA for 7 days and processed for ChIP. For ChIP, approximately 5×10⁶ cells were used per IP. ChIP assays were performed as previously described. PCR was performed using 0.1 μM of HIV-1 LTR primers (Klase et al, 2007; Guendel et al, 2009; Easley et al, 2010).

### 2. Compounds of the Invention Inhibit HIV-1 Viral Proliferation

Formula I compounds are specific and potent regulators of cell division. To that end, HIV-1 cell inhibition efficacy of several exemplary Formula I compounds was compared to commercially available cdk inhibitors.

In brief, the inhibition studies were carried out as follows. HIV-1 infected cells ACH2, J1.1, OM10.1, U1 and uninfected CEM, Jurkat T-cells, and U937 were cultured in media. The appropriate inhibitor was added at a concentration of 10 μM. Cells were treated for 48 hours and cell viability was determined using trypan blue exclusion method. Results of such a screen for 19 inhibitors is shown in Table 1 where percent dead cells are indicated each drug tested.

TABLE 1

| | | Dose | | | | | | | |
|---|---|---|---|---|---|---|---|---|---|
| Selectivity | Name | μM | ACH2 | J1.1 | OM10.1 | U1 | CEM | Jurkat | U937 |
| High | CR8 | 10 | ++++ | +++ | +++ | ++++ | + | ++ | ++ |
| | CR6 | 10 | +++ | +++ | +++ | +++ | + | ++ | ++ |
| | Meriolin4 | 10 | +++ | +++ | +++ | +++ | + | ++ | ++ |
| | Meriolin5 | 10 | +++ | ++ | ++++ | − | − | − | ++ |
| | Meriolin 6 | 10 | +++ | +++ | ++++ | − | − | − | ++ |
| Moderate | Meriolin 3 | 10 | ++ | + | − | − | − | − | − |
| | Variollin B | 10 | ++ | + | − | − | − | − | − |
| | MR3 | 10 | ++ | − | + | − | − | − | − |
| | MSL2104 | 10 | ++ | − | − | + | − | − | − |
| | MSL2106 | 10 | ++ | − | + | − | − | − | − |
| | MSL2102 | 10 | ++ | − | + | − | − | − | − |
| | MC136 | 10 | ++ | ++ | +++ | ++ | ++ | ++ | ++ |
| Poor | MSL2039 | 10 | − | − | − | − | − | − | − |
| | 1-methyl- | 10 | − | − | − | − | − | − | − |
| | 7-bromo- | | | | | | | | |
| | indirubin- | | | | | | | | |
| | 3′-oxime | | | | | | | | |
| | MC135 | 10 | − | − | − | − | − | − | − |
| | MSL2109 | 10 | − | − | − | − | − | − | − |
| | MSL2108 | 10 | − | − | − | − | − | − | − |
| | S- | 10 | − | − | − | − | − | − | − |
| | perharidine | | | | | | | | |
| | R-DRF053 | 10 | − | − | − | − | − | − | − |
| | % | − | + | ++ | +++ | ++++ | | | |
| | Inhibition | 1-5% | ~25% | ~50% | ~75% | ~90% | | | |

A number of the tested compounds caused death in HIV-1 infected cells much more efficiently than uninfected cells. The inhibitors were classified into three categories: high, moderate or poor, according to their effect on cellular viability in both HIV-1 infected and uninfected cells. Among the compounds tested in this study, CR8, a purine analog bearing a 2-pyridyl group para to the phenyl ring position 6 of the purine was the most promising drug candidate that killed HIV-1 infected cells, while having little or no effect on the death of uninfected cells.

CR8 was used by the present inventors, therefore, as a lead to synthesize a class of Formula I compounds that were found using a cell based assay to be potent cdk inhibitors. Cell inhibition data for a subset of such Formula I compounds is illustrated in Table 2.

TABLE 2

| Selectivity | Name | | ACH2 | J1.1 | OM10.1 | U1 | CEM | Jurkat | U937 |
|---|---|---|---|---|---|---|---|---|---|
| High | MRT-033 | (50 nM) | ++++ | ++ | +++ +++ | ++ + | + ++ | ++ ++ | ++++ |
| | MRT3-028 | (50 nM) | +++ | +++ | | | | | |
| Moderate | MRT2-006 | (50 nM) | + | ++ | ++ | + | − | ++ | +++ |
| | BJFP1164 | (50 nM) | ++ | ++ | ++ | + | + | ++ | ++ |
| | MRT2-004 | (50 nM) | ++ | ++ | ++ | + | + | ++ | ++ |
| | MRTI-004 | (50 nM) | ++ | ++ | ++ | + | + | ++ | ++ |
| | MRT3-007 | (50 nM) | ++ | ++ | ++ | ++ | + | + | + |
| | BJFP1155 | (50 nM) | ++ | ++ | ++ | + | + | + | + |
| | BJFP1162 | (50 nM) | ++ | ++ | ++ | + | + | + | + |
| | BJFP1167 | (50 nM) | ++ | ++ | ++ | + | + | ++ | ++ |
| Poor | MRT3-024 | (50 nM) | − | − | − | − | − | − | − |
| | BJFP1154 | (50 nM) | − | − | − | − | − | − | − |
| | MRT2-007 | (50 nM) | − | − | − | − | − | − | − |
| | MRT1-006 | (50 nM) | − | − | − | − | − | − | − |
| | BJFP1168 | (50 nM) | − | − | − | − | − | − | − |
| | MRT1-008 | (50 nM) | − | − | − | − | − | − | − |
| | MRT1-028 | (50 nM) | − | − | − | − | − | − | − |
| | MRT1-007 | (50 nM) | − | − | − | − | − | − | − |
| | Inhibition: | − | + | ++ | +++ | ++++ | | | |
| | Percentage | 1-5% | ~25% | ~50% | ~75% | ~90% | | | |

Again HIV-1 infected and uninfected cells were treated with eighteen Formula I compounds at a drug concentration of 50 nm. Forty-eight hours after treatment, cytotoxicity was determined by trypan blue dye exclusion. Percent inhibition was calculated after obtaining the cell density for each well of the tissue culture plate at the end of 48 hrs.

It was surprising for the inventors to observe that two of the tested Formula I compounds that caused maximum death in infected cells were only marginally toxic to uninfected control cells.

To evaluate if Formula I compounds that exhibit minimal inhibition of infected cells were able to inhibit viral transcription, TZM-bl cells having an integrated HIV-1 LTR-luciferase reporter were transfected with pc-Tat (encodes HIV-1 Tat protein). These cells were brought into contact with 18 Formula I compounds.

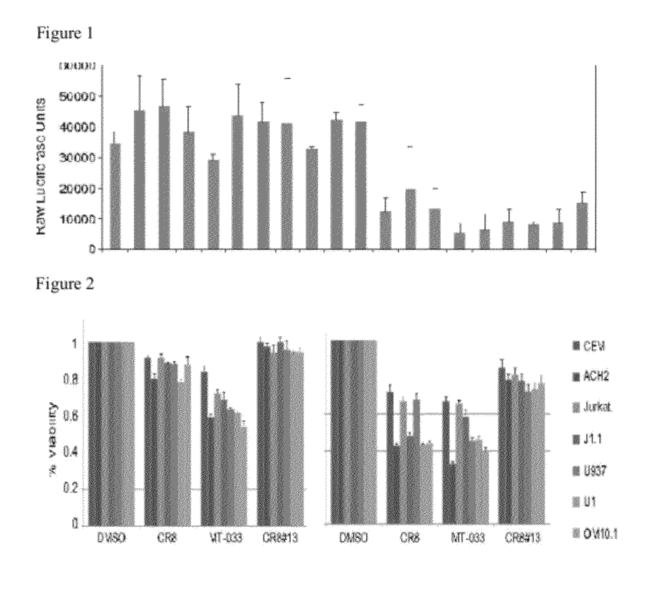

Briefly, the addition of Tat protein enables activated transcription of HIV-1 promoter driving luciferase in these cells. Treated cells were assayed at 48 hrs after Tat transfection and drug treatment. Among the tested inhibitors indicated in Table 2, the luciferase assay revealed that 9 of the Formula I compounds were efficient at decreasing viral transcription of the fully chromatinized HIV-1 promoter (FIG. 1). The Formula I compound BJFP1154 (CR8#13), exhibited the greatest transcriptional inhibition of the HIV-1 LTR, although this compound did not affect cell viability as shown in Table 2.

Quantitative MTT assays were performed (FIG. 2), to further validate the cell growth inhibitory activity of certain Formula I compounds. In this study, cultured, infected ACH2, J1.1, OM10.1, U1 and uninfected CEM, Jurkat T-cells, and U937 cells were brought in contact with Formula I compounds CR8, MRT-033, and BJFP1154 (CR8#13). Cell growth inhibition study was performed at two different concentrations of the inventive compounds, a low concentration of 50 nm, and high concentration of 10 μM.

Both CR8 and MRT-033 exhibited significant cell killing of infected cells over uninfected cells at both high and low concentrations. However, both compounds, showed some cell inhibition activity of uninfected cells. In sharp contrast, the Formula I compound BJFP1154 (CR8#13) showed marginal, if any death of uninfected cells even at the high concentration of 10 μM. Accordingly, BJFP1154 (CR8#13) represents a new class of therapeutics agent for treating and controlling HIV-1 infection.

Several other Formula I compounds also effectively decreased HIV-1 transcription without having an effect on cell viability or toxicity. For instance, while BJFP1155 and BJFP1164 show moderate cell killing activity towards infected cells, both compounds block HIV-1 transcription (FIG. 1, Lanes 16 and 18). As illustrated by data in Table 2 and FIG. 1, BJFP1154 (CR8#13) displayed very little cytotoxicity, but showed significant inhibition of HIV-1 transcription. Accordingly, Formula I compounds are a novel class of candidate therapeutics for the treatment, prevention and control of HIV-1 transcription and infection.

Efficacy Dependence of CR8#13 on microRNA Machinery for Transcription Inhibition

To evaluate the role on microRNA machinery in inhibition of viral transcription activity, HCT116 colon carcinoma cell lines that either contained a WT Dicer (HCT116 WT) or lacked the Dicer protein (HCT116 Dicer-−/−) were first transfected with the HIV-1 LTR-CAT reporter construct. The HIV-1 LTR transcription was activated with pc-Tat and following incubation for 6 hours was treated with DMSO (negative control), Flavopiridol (positive control), CR8#13, F07#13, or 9AA.

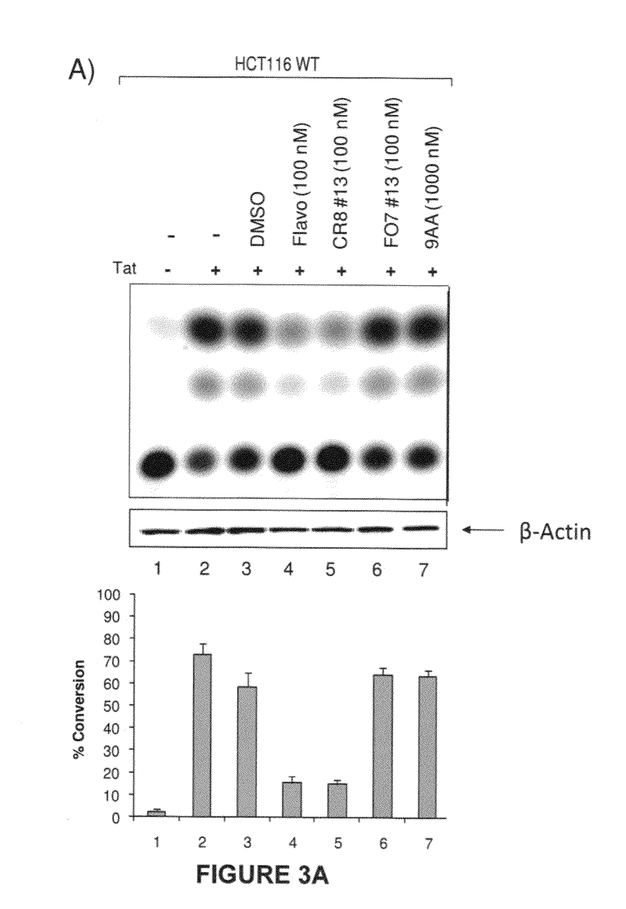

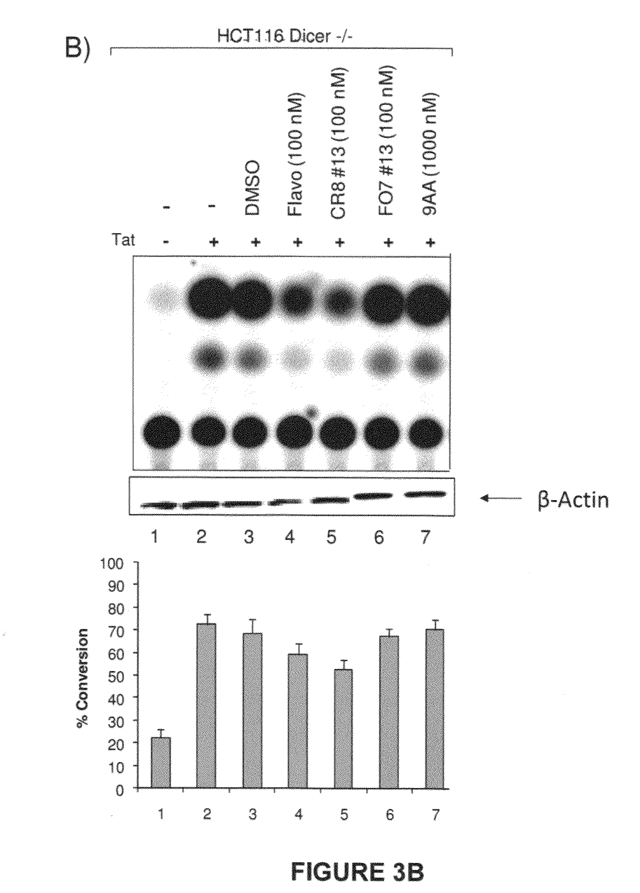

Cells were harvested 48 hrs-post treatment and processed for CAT assays (FIGS. 3A and B). As shown in this figure, there was greater activation of the HIV-1 LTR in cells lacking detectable Dicer, resulting in significantly more viral transcription than in cells containing Dicer. Based on this observation the inventors hypothesize that the microRNA machinery, Dicer and Drosha, plays an inhibitory role in HIV-1 transcription.

For instance, the inventors observed that the HIV-1 inhibitor Cyc202, effectively inhibited transcription in T-cells but not in monocytes where the microRNA machinery, Dicer and Drosha, were significantly decreased. A similar observation was made using the inventive compounds CR8#13 which inhibited viral transcription significantly better in cells that contained Dicer (FIG. 3A, Lanes 4 and 5), implicating a dependence of Formula I and II compounds on the microRNA machinery, via the RITS or RISC complex for inhibiting viral transcription.

Thus, while Flavopiridol and CR8#13 inhibited viral transcription significantly better in cells that contained Dicer (FIG. 3A, Lanes 4 and 5), F07#13 and 9AA, control drugs previously shown to inhibit viral transcription, showed only moderate inhibitory effects (FIG. 3A, Lanes 6-7). It has previously been shown that 9AA efficiently inhibited HIV-1 transcription (at higher concentrations) through the restoration of p53 and p21WAF1 functions (Guendel et al, 2009). That is, F07#13 and 9AA likely do not utilize the TAR microRNA pathway for their inhibitory activity.

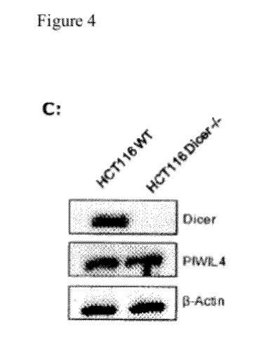

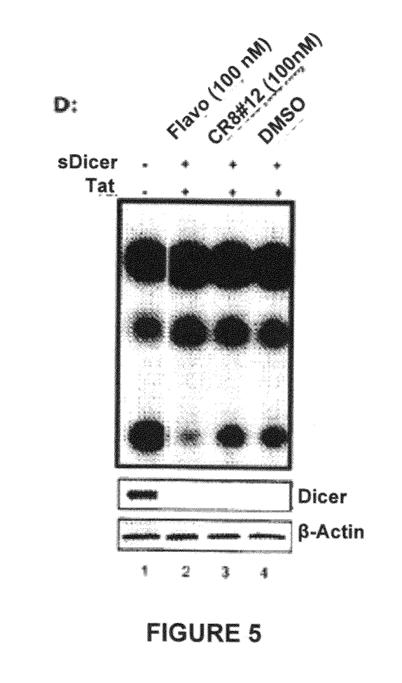

FIG. 4 substantiates that the presence of Dicer is significantly decreased in the HCT116 Dicer−/−cells. Importantly these cells contain miRNA which may be the product of PIWI expression. In order to provide further support that presence of Dicer has an effect on drug efficacy; a similar CAT assay was performed as in FIG. 3A. siRNA against Dicer was transfected along with other plasmids into HCT116 WT cells and CAT enzyme was detect in 2 days. When Dicer levels were decreased, the viral transcription inhibition caused by the drugs was also decreased (FIG. 5). The dependence of CR8#13 and Flavopiridol on Dicer for increased transcription inhibition indicated that microRNA machinery may be important in increasing efficacy and specificity toward the HIV-1 promoter.

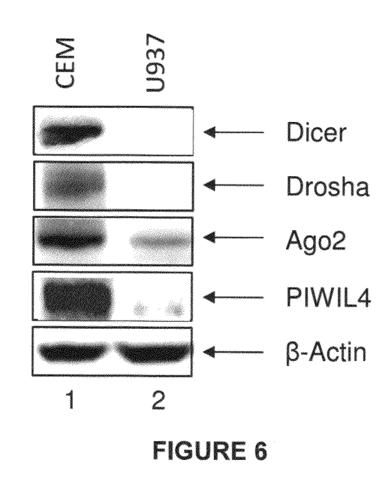

The present inventors examined whether Formula I, or Formula II compounds were effective inhibiting viral transcription in cell lines infected with an HIV-1 construct. Thus, Jurkat T-cells and promonocytic U937 cells were transfected with pNL4.3 and then treated with Flavopiridol, CR8 and CR8#13. Similar to monocytes, the Dicer levels are low to undetectable in promonocytic cells (FIG. 6) and was detectable only when these cells had differentiated into macrophages with PMA treatment.

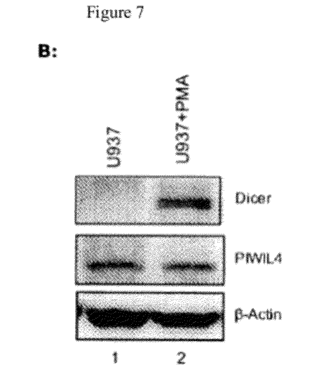

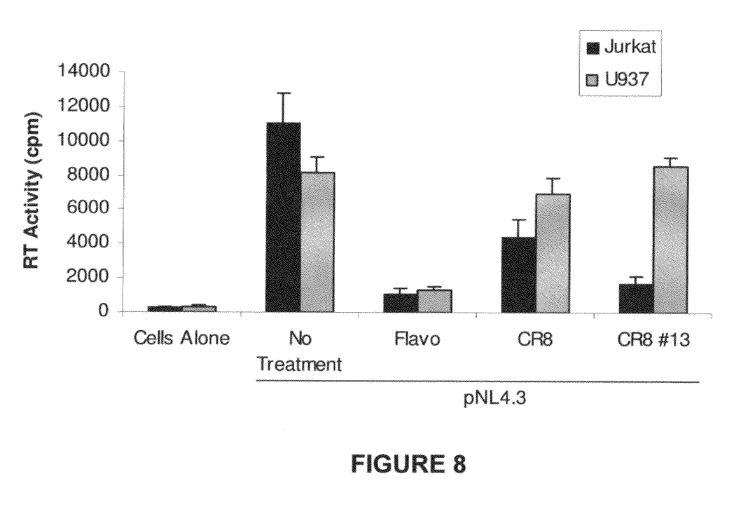

Following drug treatment cell supernatants were collected and processed for exogenous reverse transcriptase (RT) levels. As illustrated in FIG. 7, Flavopiridol was able to decrease RT levels in both cell lines. However both CR8 and CR8#13 were effective at decreasing RT levels only in the Jurkat T-cells and not U937 monocytic cells. See FIG. 8. These results further suggest the dependence of CR8#13 effects on the microRNA machinery.

CR8#13 Does Not Affect cdk9 Responsive Cellular Genes

The ultimate goal of an anti-HIV therapy is to identify compounds inhibit viral transcription at low IC₅₀ concentration with minimal, or no inhibition on cellular genes necessary for normal cell development. A determination whether inhibition cdk9 by compounds of the present invention altered the responsiveness of cdk9 dependent cellular genes, was made by the present inventor by testing the effect of CR8#13 (BJFP1154), of viral transcriptional activity utilizing a known set of cellular and viral genes. The known cdk9 responsive cellular genes used for this study were: CIITA, IL-8, CAD, MCL-1, Cyclin D1, and PBX-1 and Histone H2B gene served as a negative control since this gene does not rely on the activity of cdk9 its expression (See, Medlin et al, 2005).

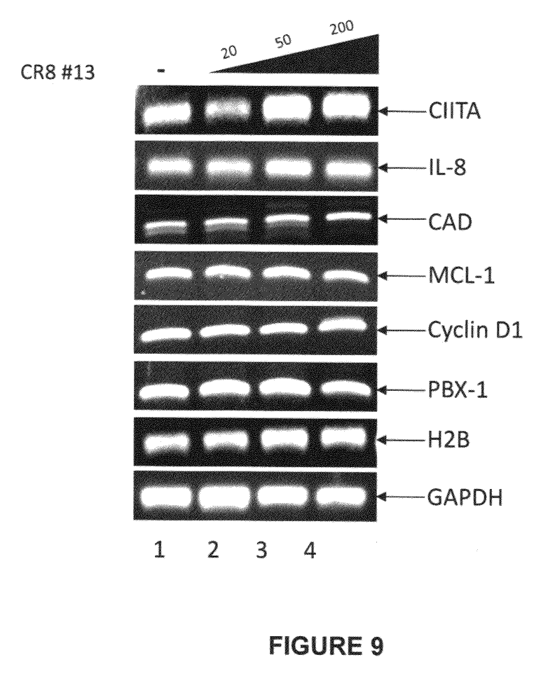

Results in FIG. 9 show that treatment of cells with CR8#13 at various concentrations (20, 50, 200 nm) did not inhibit transcription of genes that are cdk9 responsive. This data reinforces the hypothesis that the transcriptional inhibition caused by CR8#13 may be specific to HIV-1 promoter and not cellular genes that utilize cdk9 pathway.

Possible Effect of TAR microRNA in Increasing the Effectiveness of HIV-1 Transcription Inhibitors

Further studies aimed at evaluating the molecular mechanism of inhibition of HIV-1 transcription by compounds according to the present invention focused on the effect of TAR microRNA in enhancing the potency of transcriptional inhibition. Briefly, latently infected cells were analyzed for the presence of short, abortive RNA transcripts, approximately 50-100 nt in length, that contain the HIV TAR stemloop. Previous studies have shown that TAR serves as a substrate for Dicer and cleaves the RNA transcripts to short a 21-22 nucleotide RNA molecule that is capable of silencing HIV-1 transcription.

Since these TAR-containing short transcripts are the dominant HIV-1 RNA produced in appreciable quantities during latency, it was hypothesized by the inventor that these RNA molecules could suppress viral gene expression. Thus, cdk inhibitors of the present invention suppress HIV-1 transcription by functionally interacting with the TAR microRNA, to alter the activity of RNA polymerase II and ultimately cause inhibition on the HIV-1 promoter.

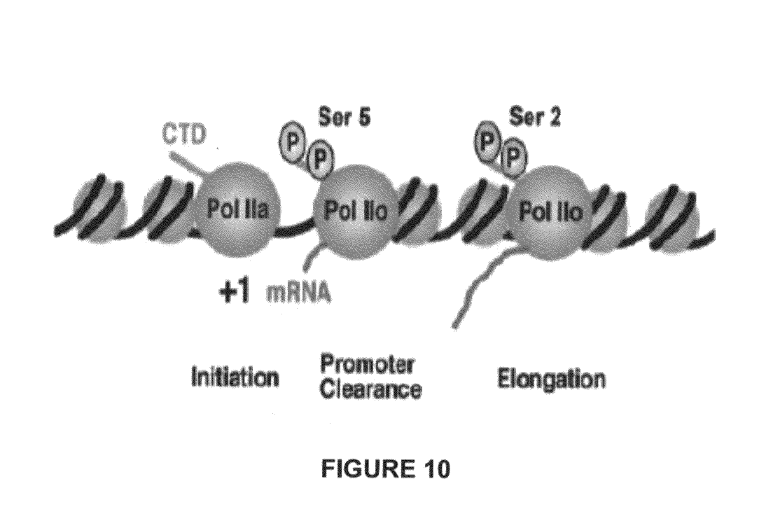

As seen in FIG. 10, RNA polymerase II is phosphorylated at Ser5 in the initiation complex and Ser2 in the elongation complex. RNA polymerase II associated with HIV-1 promoter, however, is phosphorylated on both Ser2 and 5 in the presence of Tat (see Zhou et al, 2004). In order to ascertain whether or not cells treated with CDK inhibitors according to the present invention produce additional TAR derived miRNA, the inevntor utilized RNase protection assays to detect small RNA fragments corresponding to the TAR sequence.

Briefly, a probe complementary to the entire length of the 5′ portion of the TAR stem loop was designed, which would detect the generation of ˜21 nt RNAs from any position within that sequence. The results were considered positive if the 32 nt probe was cleaved to ˜21 nt, indicating protection by a microRNA. The latently infected T-cell clone, ACH2, was treated with TNF (for viral induction) followed by treatment with the cdk inhibitor Cyc202, CR8, and a compound according to the present invention (CR8#13).

Thirty micrograms of total RNA from each condition was used for RPA analysis used to detect the presence of the 5′ TAR miRNA. Higher levels of TAR and miTAR RNA were observed in CR8#13 treated cells as compared to Flavopiridol treatment (FIG. 11, Lanes 5 and 6). This lead to further experimentation to determine levels of each of the microRNA produced from TAR region (3′TAR stem and 5′TAR stem) in presence of these drugs.

Total RNA from the ACH2 cells treated with Flavopiridol, CR8, and CR8#13 were extracted and processed for RTPCR detection of microRNAs, specifically for the 3′TAR and 5′TAR stems regions. The QuantiMir RT Kit provides a simple and sensitive method to detect small RNA molecules. Following extraction of total RNA, the microRNAs are polyA tagged and then an oligo-dT adaptor is annealed. Reverse transcriptase was used to create cDNAs. The standard end-point PCR can be used to detect specific microRNAs. The product obtained contains an adaptor (46 bp) plus the miRNA (˜2 bp).

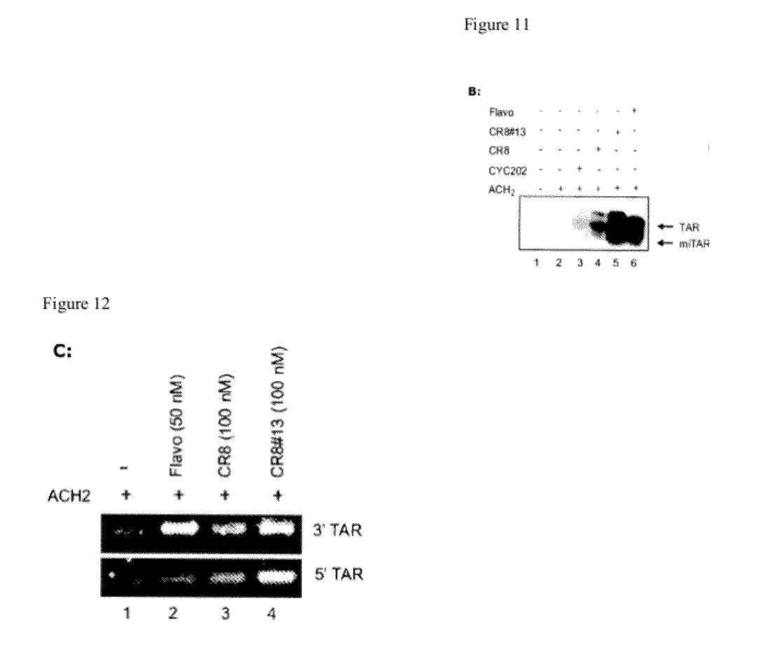

As illustrated in FIG. 12 there is an increase in 3′TAR with each drug treatment over the untreated control. Flavopiridol and CR8#13 treatment exhibited a similar increase in 3′TAR microRNA levels. In contrast, there was an increase in the 5′TAR microRNA levels only with the cells treated with CR8#13. These observations (FIG. 12) could potentially explain the higher levels of miTAR observed in FIG. 11 (Lane 5).

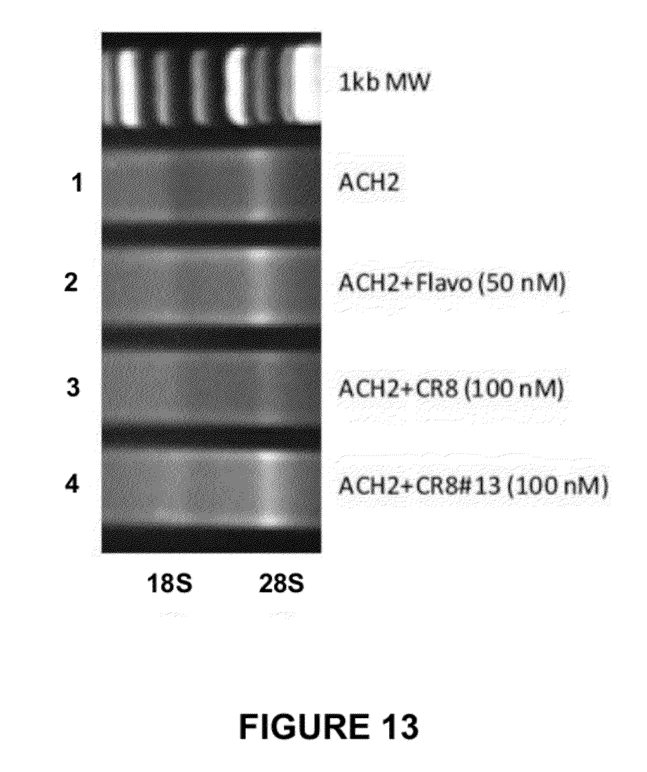

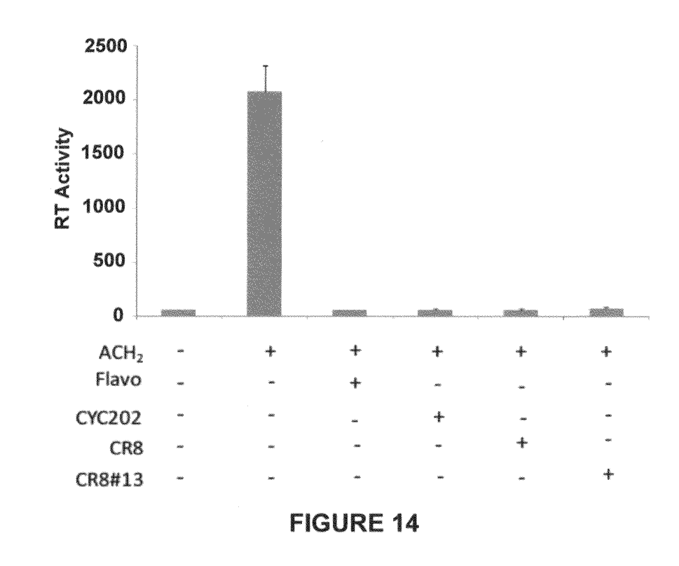

The integrity of the total RNA was confirmed by agarose gel to check for integrity of the total RNA (FIG. 13). Based on levels of reverse transcriptase in the supernatant of drug treated cells it was concluded that almost complete inhibition of virus replication occurs in these cells (FIG. 14). Collectively, these results led the inventor to hypothesize that the 5′TAR microRNA could potentially be responsible for the effective inhibitory effects of CR8#13, suggesting that inhibitors of RNA polymerase II Ser2/Ser5 phosphorylation may slow down movement of RNA polymerase II toward the 3′ end of the HIV-1 genome so as to create more short TAR transcripts than normally present in these cells, thus providing a mechanistic explanation of transcription inhibition observed with inhibitors, such as CR8#13.

TAR microRNA Induce Formation of Repressive Chromatin Markers on the HIV-1 LTR

Recent studies have suggested that TAR derived microRNA may have effects on chromatin structure (Klase et al, 2009). To test the ability of TAR derived microRNA to direct chromatin remodeling at the viral LTR, chromatin immunoprecipitation (ChIP) assays were performed by the inventor to examine the recruitment of factors to the HIV-1 LTR. One such factor, HDAC-1, is a histone deacetylase which is implicated to be involved in silencing of HIV-1 promoter.

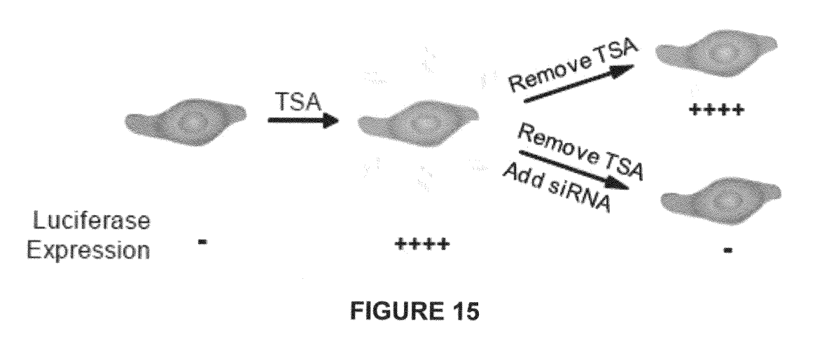

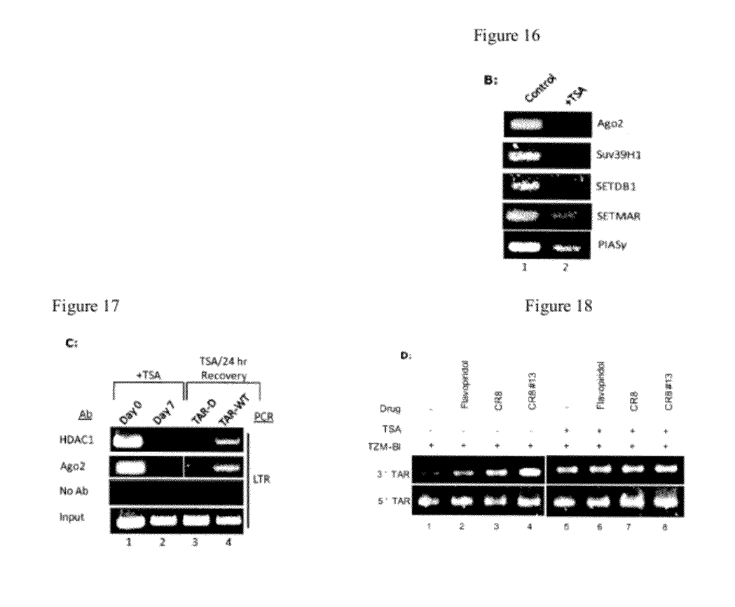

Accordingly, TZM-bl cells carrying integrated HIV-1 LTR were utilized (FIG. 15), since LTR is already silenced in these cells. Cells were treated for seven days with a sub-lethal dose of the HDAC inhibitor TSA and assayed for factor occupancy on the promoter. Chromatin changes were verified by performing ChIP assays before and after TSA treatment using antibodies specific for inhibitory factors including the components of the RNAi machinery (FIG. 16). This study evaluated whether cells in the silenced state (absence of TSA) would have differing factor occupancy as compared to active state (presence of TSA). The results in panel B established that the RNAi protein Argonaute and the histone modifiers Suv39H1 and SETDB1 are present at the latent LTR and are removed once they are treated with TSA.

To evaluate the effect of TAR microRNA on recruitment of repressive chromatin remodeling factors to the HIV-1 LTR, the inventors treated TZM-bl cells with TSA for 7 days followed by removal of TSA and transfection of these cells with either TAR-WT or TAR-D RNA control. Following treatment with TSA, on day 7, the cell culture medium was removed and replaced with complete media. The following day (day 8), cells were analyzed by the ChIP assay to for presence of either HDAC-1 and/or Argonaute (Ago2) (FIG. 17). As illustrated in FIG. 18, prior to TSA treatment HDAC-1 and Ago2 were associated with the LTR and this association was lost upon treatment with TSA (compare lanes 1 and 2). Transfection of the cells with TAR-WT RNA led to an increase in the re-association of HDAC-1 and Ago2 to the LTR after 24 hrs as compared to the control RNA (compare lane 3 to 4). HDAC-1 and Ago2 recruitment to an integrated LTR verifies that heterochromatin formation at the HIV-1 LTR is driven by RNAi mediated TAR microRNA.

To determine whether the cdk inhibitors Flavopiridol, CR8, and CR8#13 increased levels of TAR microRNA in the TSA-treated and control TZM-bl cells total RNA was extracted from both TSA treated and control cells treated with Flavopiridol, CR8, and CR8#13 and processed for RT-PCR detection of microRNAs, specifically, for the presence of 3′TAR and 5′TAR molecules.

As illustrated in FIG. 18 both 3′ and 5′ TAR microRNA's are present in these cells. The TZMbl cells treated with TSA expressed higher amounts of both 3′ and 5′ TAR microRNA when treated with Flavopiridol and CR8. The addition of Flavopirodol, CR8, and especially CR8#13 significantly increased amount of the 3′ TAR microRNA in the non-TSA treated TZM-bl cells. This was expected since previous results have shown that integral microRNA machinery (i.e., Ago2) can be found near the HIV-1 LTR before TSA treatment. A similar increase in the 3′ and 5′TAR microRNA was observed from cells that had been treated first with TSA and then CR8#13.

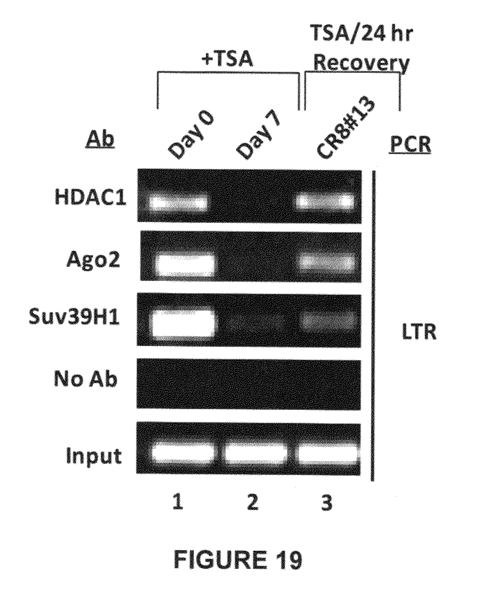

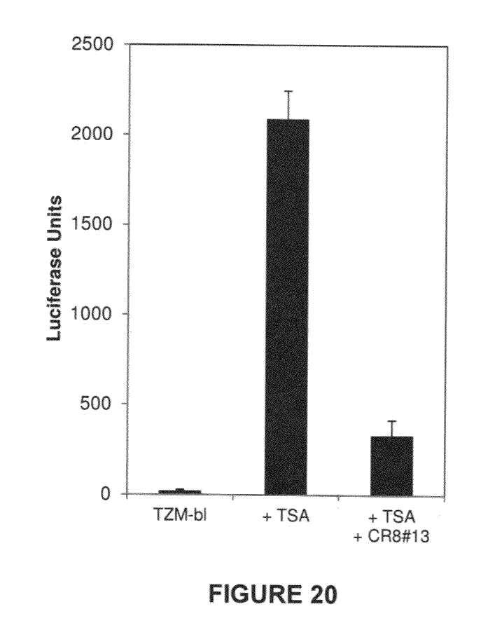

To observe the effect of drug on heterochrmatin formation, the present inventor used a ChIP assay. Briefly, TZM-bl cells were treated with TSA (FIGS. 16 and 17), and then treated with CR8#13. As illustrated in FIG. 19 treatment with CR8#13 results in the re-recruitment of heterochromatin markers, such as HDAC1, Ago2, and Suv39Hl to the HIV-1 promoter. Additionally, a Luciferase based assay using TSA and CR8#13 treated cells showed that treatment with TSA alone increased Luciferase activity of the intergrated HIV-1-Luc in TZM-bl cells, while treatment of cells using both TSA and CR8#13 caused a decrease Luciferase activity. See FIG. 20.

Collectively, these results indicate that the HIV-1 TAR microRNA is responsible for the enhanced effectiveness of cdk inhibitors, especially CR8#13, on the HIV-1 promoter by recruiting both microRNA and chromatin remodeling complexes to the promoter proximal region.

The above results and data illustrated in the figures indicate that compounds according to the present invention are potent inhibitors of cdk and are candidate drugs for inhibiting HIV-1 transcription and treating HIV-1 infection. Without being bound to a particular theory, at the molecular level, expression of Dicer is required for down regulating or inhibiting viral transcription by the inventive cdk inhibitors. This hypothesis stems from the observation that the inventive compounds have improved efficacy in T-cells that express higher levels of Dicer than monocytes in which the expression levels of Dicer is much lower leading the inventor to conclude that viral TAR microRNA plays a role in drug efficacy.

Stated differently, while both Flavopiridol and CR8#13 exhibit a dependence on the presence of the viral TAR microRNA, the exhibited down regulation of viral transcription by both drugs was significantly more when Dicer is present to increase the cellular levels of viral TAR microRNA. Indeed, the greater potency of CR8#13 in comparison to Falvopiridol, is most likely due to the increased production of viral TAR microRNA in CR8#13 treated infected cells.

CR8#13 was also observed to promote a significant increase in both the 3′TAR and 5′TAR microRNA's, while Flavopiridol only increased the 3′TAR microRNA. To understand mechanistically how the TAR microRNA inhibits HIV-1 transcription, the present inventor used TZM-bl cells. Experimental evidence from the inventor's study showed that t chromatin remodeling complexes and microRNA machinery are not bound to the HIV-1 LTR when the LTR becomes activated. However, when the TAR microRNA is reapplied, these complexes are re-linked to the LTR to terminate RNA polymerase II transcription while creating more TAR microRNA.

Overall inhibition of HIV-1 replication by a compound according to this invention (CR8#13), most likely is due to the production of both the 3′ and 5′ TAR microRNA , the premature termination of RNA polymerase II by inhibiting Pol II phosphorylation and elongation and recruitment of microRNA machinery to the HIV-1 LTR.

Overall, low concentrations of a cdk inhibitor according to the present invention will result in inhibition of Pol II phosphorylation and elongation, increase TAR microRNA levels and result in a higher specificity of inhibition toward the HIV-1 promoter as compared to other cellular or viral promoters.

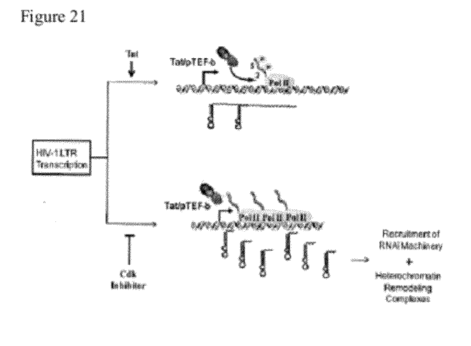

Graphically, FIG. 21 shows a model for cdk-mediated viral microRNA production and inhibition of HIV-1 gene transcription. As illustrated in this figure cdk inhibitors of the present invention reduce phosphorylation of RNA polymerase II which ultimately inhibits viral transcription.

What is claimed is:

1. A process for inhibiting the replication of human immunodeficiency virus-1 (HIV-1), comprising contacting a cell with at least one compound according to Formula I, 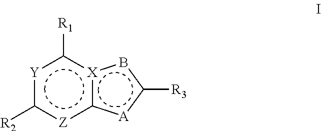
wherein
R₁ and R₂ are each independently selected from the group consisting of —H, straight or branched chain (C₁-C₆)alkyl, straight or branched chain (C₁-C₆)hydroxyalkyl, (C₂-C₆)alkene, (C₃-C₈)cycloalkyl, (C₃-C₁₄)aryl, halogen, —NR^{a}R^{b}, —NR^{a}(C₁-C₆)hydroxyalkyl, —NR^{a}(C₁-C₆)alkylene-(C₃-C₁₄)aryl, —NR^{a}(C₁-C₆)alkylene-(C₃-C₁₄)heteroaryl, —NR^{a}(C₁-C₆)alkylene-(C₃-C₁₄)heterocycloalkyl, —NR^{a}(C₁-C₆)alkylene-(C₃-C₁₄)cycloalkyl, —OR^{c}, and —SR^{c};
R₃ is selected from the group consisting of hydrogen, straight or branched chain (C₁-C₆)alkyl, —OH and halogen;
X, Y, Z, A and B are each independently selected from the group consisting of a bond,
—C(R′″)₂—, —CR′″—, —NR′″—, —N—, —O—, —C(O)—, and —S—, wherein
no more than three of X, Y, Z, A and B simultaneously represent a bond; and
X and B are not simultaneously —O—, or —S—;
each [symbol] represents the option of having one or more double bonds;
R^{a}, R^{b}, R^{c} and R′″ are each independently selected from the group consisting of H, OH, straight or branched chain (C₁-C₈)alkyl, (C₃-C₆)aryl, —NH₂, —C(O)(C₁-C₆)alkyl, —C(O)(C₃-C₁₄)aryl, (C₃-C₆)cycloalkyl, (C₃-C₁₄)heterocycloalkyl, (C₃-C₁₄)heteroaryl, (C₃-C₁₄)aryl-(C₁-C₆)alkylene-, (C₃-C₆)cycloalkyl-(C₁-C₆)alkylene-, (C₃-C₁₄)heteroaryl-(C₁-C₆)alkylene-, (C₃-C₁₄)heterocycloalkyl-(C₁-C₆)alkylene-; and
wherein any alkyl, alkylene, alkene, aryl, heteroaryl, cycloalkyl, or heterocycloalkyl is optionally substituted with one or more members selected from the group consisting of halogen, oxo, —COOH, —CN, —NO₂, —OH, straight or branched chain (C₁-C₆)alkyl, (C₂-C₆)alkene, (C₃-C₁₄)aryl, (C₃-C₁₄)heteroaryl, (C₃-C₁₄)heterocycloalkyl, (C₃-C₁₄)cycloalkyl, (C₁-C₆)alkoxy, and (C₃-C₁₄)aryloxy;
or a pharmaceutically acceptable salt, stereoisomer, tautomer, or prodrug thereof.

2. The process according to claim 1, wherein X, Y, Z and B are —N—, A is C(R′″), R₃ is hydrogen and [symbol] represents the option of having one or more double bonds.

3. The process according to claim 2, wherein R′″ is a straight or branched chain (C₁-C₆)alkyl.

4. The process according to claim 3, wherein R′″ is isopropyl.

5. The process according to claim 1, wherein R₁ is NR^{a}(C₁-C₆)alkylene-(C₃-C₁₄)aryl.

6. The process according to claim 5, wherein R₁ is —NH—(CH₂)-phenyl.

7. The process according to claim 6, wherein the phenyl is further substituted by a (C₃-C₁₄)heteroaryl.

8. The process according to claim 7, wherein the (C₃-C₁₄)heteroaryl is a pyridine.

9. The process according to claim 1, wherein R₂ is —NR^{a}(C₁-C₆)hydroxyalkyl.

10. The process according to claim 9, wherein R^{a} is —H.

11. The process according to claim 1, wherein the compound is selected from the following table:

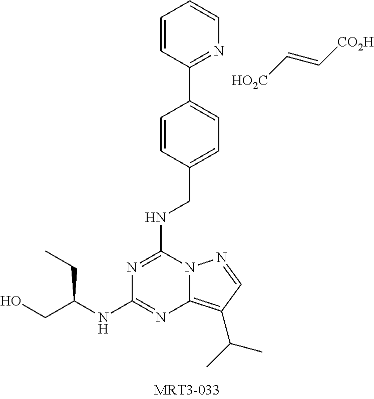

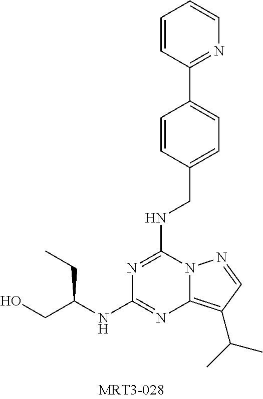

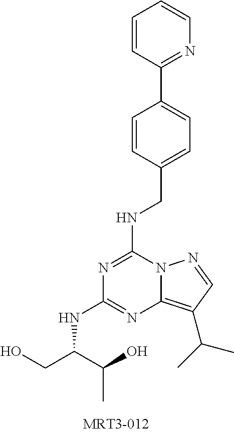

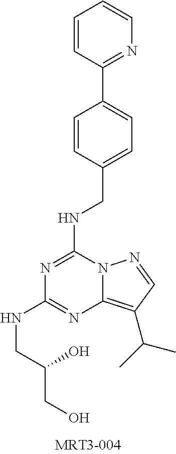

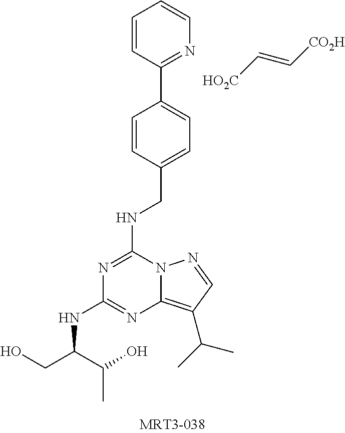

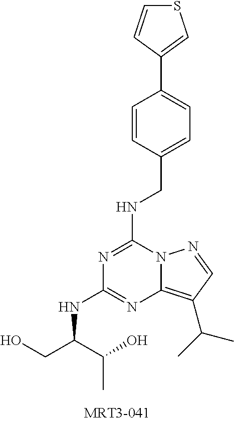

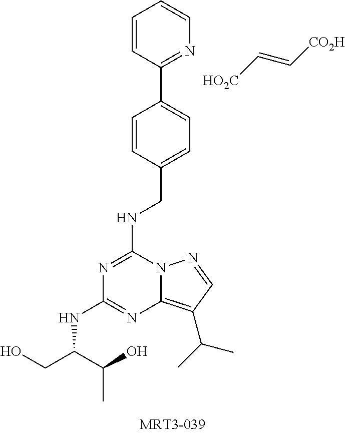

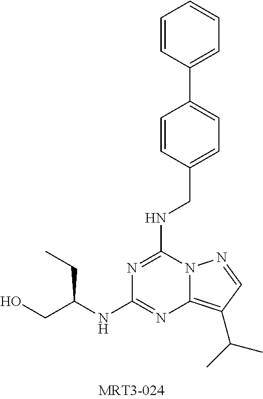

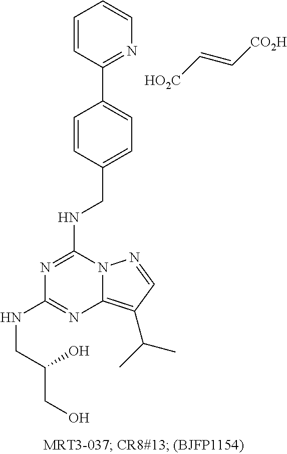

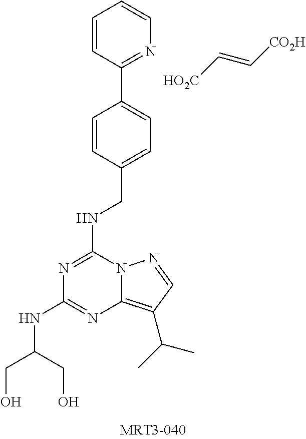

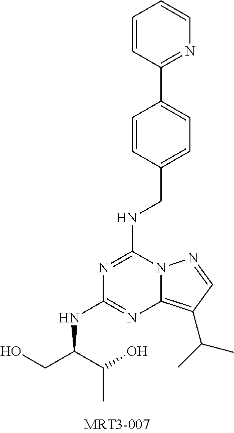

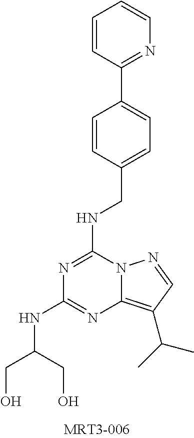

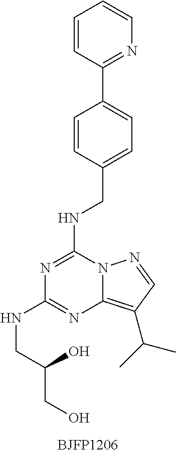

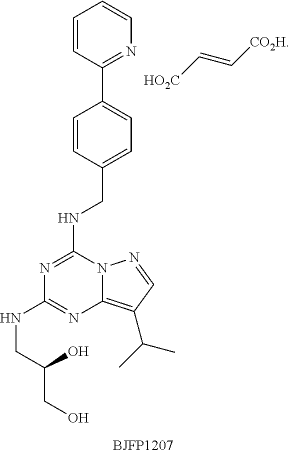

12. A method for the treatment or prevention of a HIV-1 infection in a subject, comprising administering to the subject therapeutically effective amount of at least one compound according to Formula I, 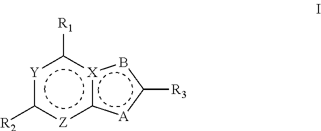
wherein
R₁ and R₂ are each independently selected from the group consisting of —H, straight or branched chain (C₁-C₆)alkyl, straight or branched chain (C₁-C₆)hydroxyalkyl, (C₂-C₆)alkene, (C₃-C₈)cycloalkyl, (C₃-C₁₄)aryl, halogen, —NR^{a}R^{b}, —NR^{a}(C₁-C₆)hydroxyalkyl, —NR^{a}(C₁-C₆)alkylene-(C₃-C₁₄)aryl, —NR^{a}(C₁-C₆)alkylene-(C₃-C₁₄)heteroaryl, —NR^{a}(C₁-C₆)alkylene-(C₃-C₁₄)heterocycloalkyl, —NR^{a}(C₁-C₆)alkylene-(C₃-C₁₄)cycloalkyl, —OR^{c}, and —SR^{c};
R₃ is selected from the group consisting of hydrogen, straight or branched chain (C₁-C₆)alkyl, —OH and halogen;
X, Y, Z, A and B are each independently selected from the group consisting of a bond,
—C(R′″)₂—, —CR′″—, —NR′″—, —N—, —O—, —C(O)—, and —S—, wherein
no more than three of X, Y, Z, A and B simultaneously represent a bond; and
X and B are not simultaneously —O—, or —S—;
each [symbol] represents the option of having one or more double bonds;
R^{a}, R^{b}, R^{c} and R′″ are each independently selected from the group consisting of H, OH, straight or branched chain (C₁-C₈)alkyl, (C₃-C₆)aryl, —NH₂, —C(O)(C₁-C₆)alkyl, —C(O)(C₃-C₁₄)aryl, (C₃-C₆)cycloalkyl, (C₃-C₁₄)heterocycloalkyl, (C₃-C₁₄)heteroaryl, (C₃-C₁₄)aryl-(C₁-C₆)alkylene-, (C₃-C₆)cycloalkyl-(C₁-C₆)alkylene-, (C₃-C₁₄)heteroaryl-(C₁-C₆)alkylene-, (C₃-C₁₄)heterocycloalkyl-(C₁-C₆)alkylene-; and
wherein any alkyl, alkylene, alkene, aryl, heteroaryl, cycloalkyl, or heterocycloalkyl is optionally substituted with one or more members selected from the group consisting of halogen, oxo, —COOH, —CN, —NO₂, —OH, straight or branched chain (C₁-C₆)alkyl, (C₂-C₆)alkene, (C₃-C₁₄)aryl, (C₃-C₁₄)heteroaryl, (C₃-C₁₄)heterocycloalkyl, (C₃-C₁₄)cycloalkyl, (C₁-C₆)alkoxy, and (C₃-C₁₄)aryloxy;
or a pharmaceutically acceptable salt, stereoisomer, tautomer, or prodrug thereof.

13. The method according to claim 12, wherein the compound is selected from the following table:

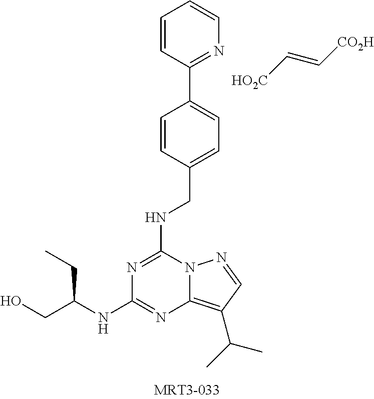

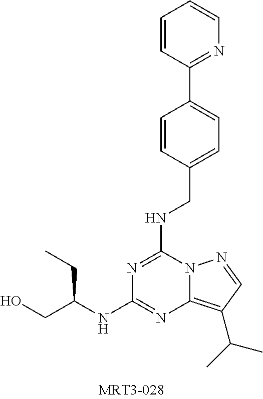

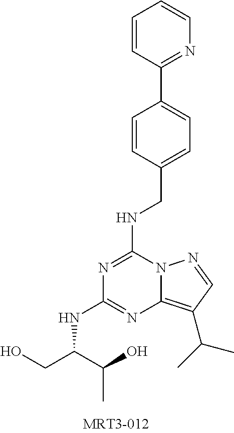

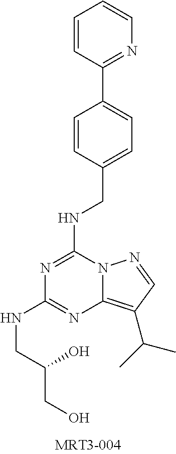

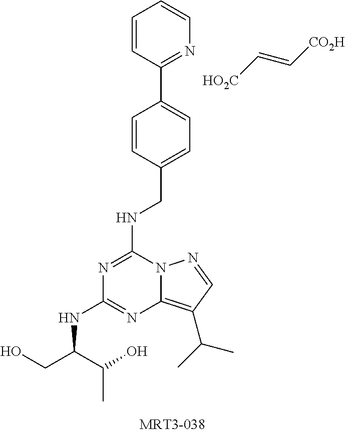

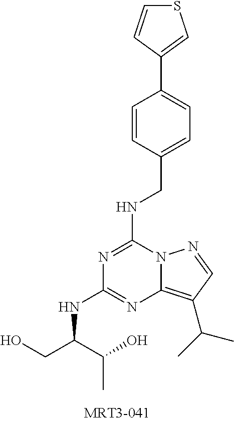

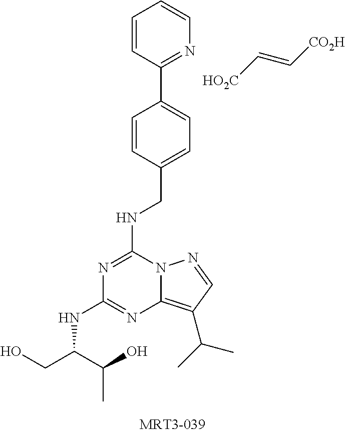

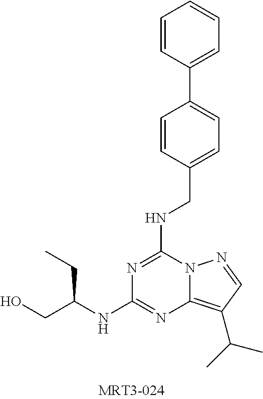

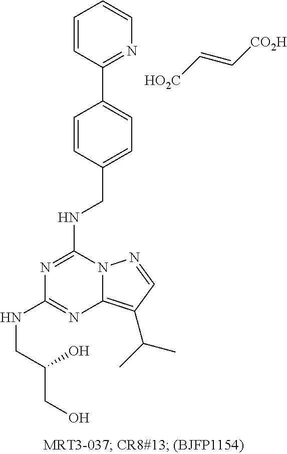

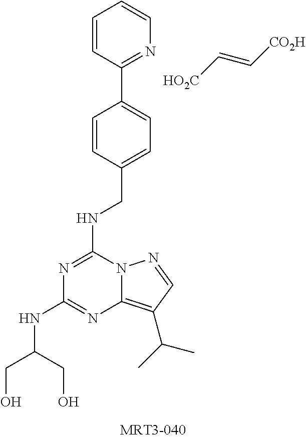

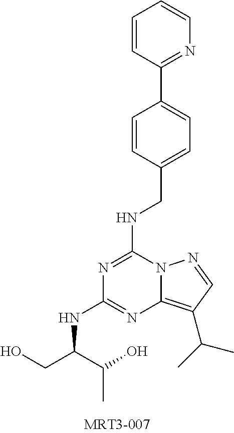

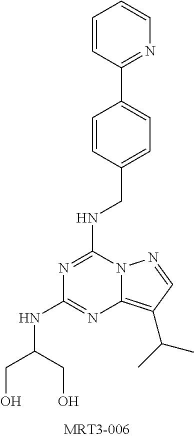

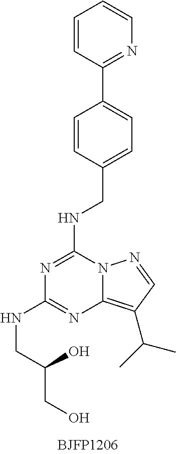

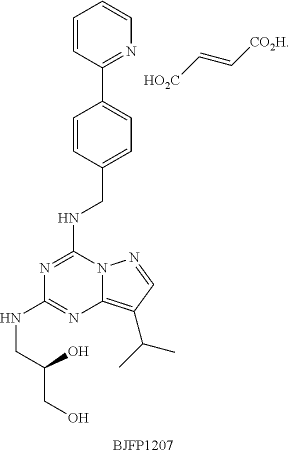

14. A method for modulating the activity of a cyclin dependent kinase (cdk) in a cell infected with HIV-1, comprising contacting the cell with a therapeutically effective amount of at least one compound according to Formula I, 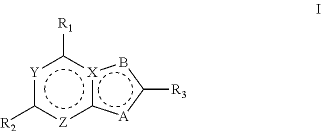
wherein
R₁ and R₂ are each independently selected from the group consisting of —H, straight or branched chain (C₁-C₆)alkyl, straight or branched chain (C₁-C₆)hydroxyalkyl, (C₂-C₆)alkene, (C₃-C₈)cycloalkyl, (C₃-C₁₄)aryl, halogen, —NR^{a}R^{b}, —NR^{a}(C₁-C₆)hydroxyalkyl, —NR^{a}(C₁-C₆)alkylene-(C₃-C₁₄)aryl, —NR^{a}(C₁-C₆)alkylene-(C₃-C₁₄)heteroaryl, —NR^{a}(C₁-C₆)alkylene-(C₃-C₁₄)heterocycloalkyl, —NR^{a}(C₁-C₆)alkylene-(C₃-C₁₄)cycloalkyl, —OR^{c}, and —SR^{c};
R₃ is selected from the group consisting of hydrogen, straight or branched chain (C₁-C₆)alkyl, —OH and halogen;
X, Y, Z, A and B are each independently selected from the group consisting of a bond,
—C(R′″)₂—, —CR′″—, —NR′″—, —N—, —O—, —C(O)—, and —S—, wherein
no more than three of X, Y, Z, A and B simultaneously represent a bond; and
X and B are not simultaneously —O—, or —S—;
each [symbol] represents the option of having one or more double bonds;
R^{a}, R^{b}, R^{c} and R′″ are each independently selected from the group consisting of H, OH, straight or branched chain (C₁-C₈)alkyl, (C₃-C₆)aryl, —NH₂, —C(O)(C₁-C₆)alkyl, —C(O)(C₃-C₁₄)aryl, (C₃-C₆)cycloalkyl, (C₃-C₁₄)heterocycloalkyl, (C₃-C₁₄)heteroaryl, (C₃-C₁₄)aryl-(C₁-C₆)alkylene-, (C₃-C₆)cycloalkyl-(C₁-C₆)alkylene-, (C₃-C₁₄)heteroaryl-(C₁-C₆)alkylene-, (C₃-C₁₄)heterocycloalkyl-(C₁-C₆)alkylene-; and
wherein any alkyl, alkylene, alkene, aryl, heteroaryl, cycloalkyl, or heterocycloalkyl is optionally substituted with one or more members selected from the group consisting of halogen, oxo, —COOH, —CN, —NO₂, —OH, straight or branched chain (C₁-C₆)alkyl, (C₂-C₆)alkene, (C₃-C₁₄)aryl, (C₃-C₁₄)heteroaryl, (C₃-C₁₄)heterocycloalkyl, (C₃-C₁₄)cycloalkyl, (C₁-C₆)alkoxy, and (C₃-C₁₄)aryloxy;
or a pharmaceutically acceptable salt, stereoisomer, tautomer, or prodrug thereof.

15. The method according to claim 14, wherein the cyclin dependent kinase is selected from the group consisting of cdk1, cdk2, cdk5 and cdk9.

16. The method according to claim 14, wherein the compound is selected from the following table: 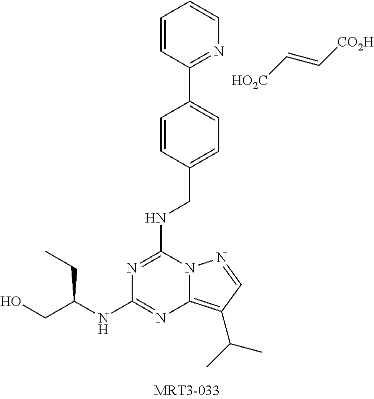 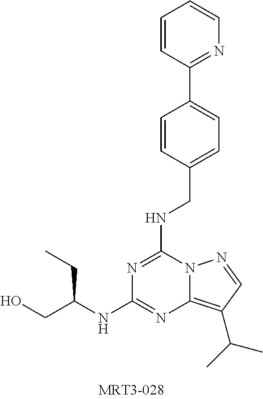 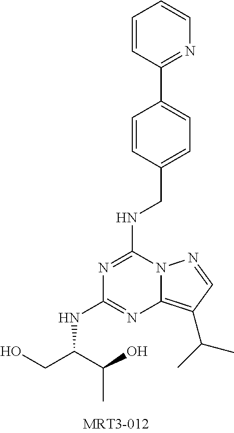 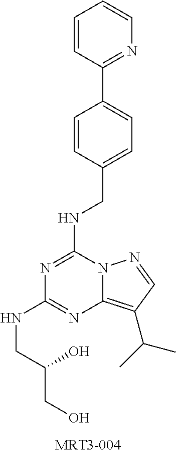 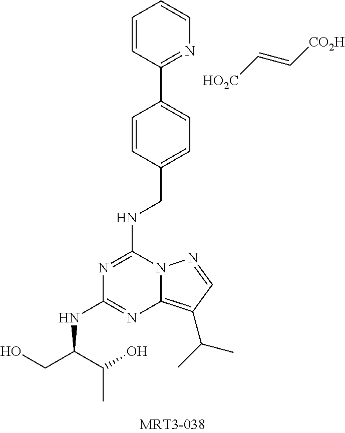 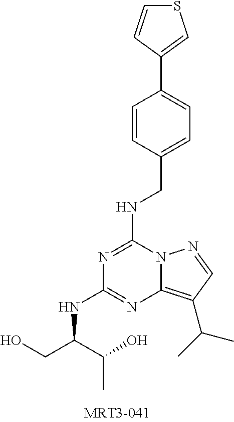 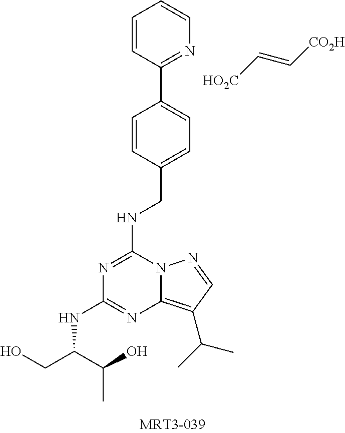 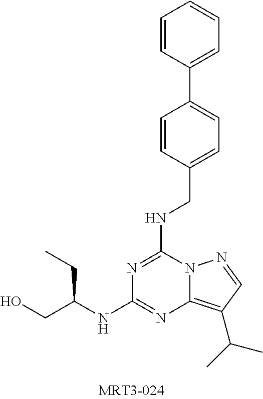 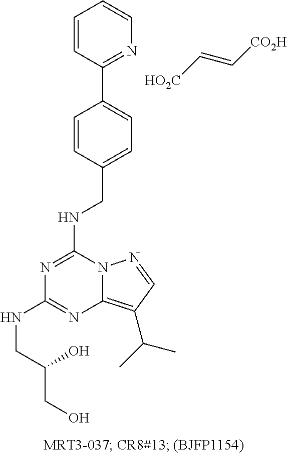 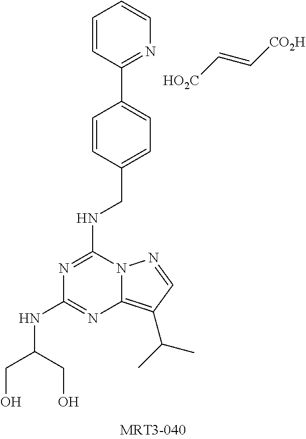 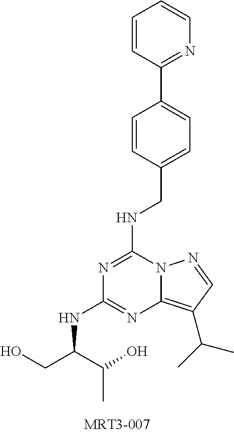 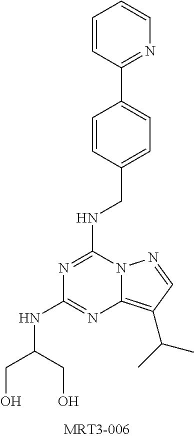 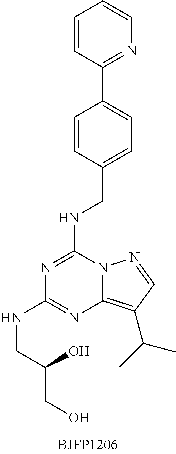 [chemical structure]
